(19) United States
(12) Patent Application Publication
(10) Pub. No.: US 2026/0109930 A1
(43) Pub. Date: Apr. 23, 2026

(54) METHODS OF OPTIMIZING DISSOLVED OXYGEN LEVELS IN CELL CULTURE

(71) Applicant: Genzyme Corporation, Cambridge, MA (US)

(72) Inventors: Ethan Disston Penner, Cambridge, MA (US); Charles Budde, Cambridge, MA (US); Jeffrey Swana, Cambridge, MA (US)

(73) Assignee: Genzyme Corporation, Cambridge, MA (US)

(21) Appl. No.: 19/118,698

(22) PCT Filed: Oct. 6, 2023
(86) PCT No.: PCT/US2023/034679

§ 371 Date: Apr. 4, 2025

Related U.S. Application Data
(60) Provisional application No. 63/414,691, filed on Oct. 10, 2022.

(51) Int. Cl. C12M 1/34; C12M 1/00; C12N 5/071

(52) U.S. Cl. CPC C12M 41/34; C12M 29/10; C12M 41/12; C12M 41/32; C12M 41/36; C12M 41/44; C12N 5/0602

(57) ABSTRACT

Provided herein are methods of selecting an optimal fixed orientation of a dissolved oxygen (DO) probe in a perfusion bioreactor and culturing a mammalian cell in a perfusion bioreactor having the DO probe positioned at the selected fixed orientation.

### CROSS-REFERENCE TO RELATED APPLICATION

This application claims priority to U.S. Provisional Patent Application Ser. No. 63/414,691, filed Oct. 10, 2022; the entire contents of which are herein incorporated by reference.

### TECHNICAL FIELD

The present disclosure relates to methods of accurately measuring the level of dissolved oxygen (DO) in a perfusion bioreactor for cell culture. The accurate measurement of DO levels allows the optimization of culturing conditions such as DO level and volumetric input power.

### BACKGROUND

Dissolved oxygen measurement is essential in fermentation and cell culture for ensuring conditions remain optimal for cells. Low dissolved oxygen levels in fermenters/bioreactors can impact growth rate, nutrient uptake, cellular morphology, and metabolite synthesis; leading to reduced yield and lower end-product quality. High DO levels cause the formation of reactive oxygen species that can oxidize components in the medium and result in cell mutations. Further, in large fermenters that require powerful compressors to inject air, excessive sparging is a costly waste of energy. Maintaining dissolved oxygen in the required range is therefore critical for process optimization. Accurate oxygen control is therefore critical and can be achieved only if measurements from dissolved oxygen sensors installed in fermenters/bioreactors are reliable and accurate.

DO control is essential for maintenance of proper aerobic metabolism in mammalian cell culture but can be difficult in intensified perfusion processes due to high oxygen demand and culture viscosity. Therefore, methods for accurate measurement of DO levels in benchtops and refinements of the pilot scale operation are needed for a successful process scale-up.

### SUMMARY

Provided herein are methods for culturing a mammalian cell and selecting a fixed orientation of a DO probe in a perfusion bioreactor to allow the accurate measurement of the DO level in the culture.

Also provided herein are methods of culturing a mammalian cell in a perfusion bioreactor that include: (a) placing a dissolved oxygen (DO) probe in the perfusion bioreactor comprising a liquid at a plurality of different fixed orientations; (b) measuring the DO level using the DO probe at each of the plurality of different fixed orientations; (c) selecting a fixed orientation for the DO probe in the bioreactor based on the measured DO levels in step (b), wherein the selected fixed orientation does not demonstrate measurement of falsely increased DO levels as determined by analysis of the measured DO levels in step (b); and (d) culturing a mammalian cell in a liquid culture medium in the perfusion bioreactor having the DO probe positioned at the selected fixed orientation.

Also provided herein are methods of selecting a fixed orientation for a dissolved oxygen (DO) probe in a perfusion bioreactor that include: (a) placing the DO probe in the perfusion bioreactor comprising a liquid at a plurality of different fixed orientations; (b) measuring the DO level using the DO probe at each of the plurality of different fixed orientations; and (c) selecting a fixed orientation for the DO probe in the bioreactor based on the measured DO levels in step (b), wherein the selected fixed orientation does not demonstrate measurement of falsely increased DO levels as determined by analysis of the measured DO levels in step (b).

Some embodiments of any of the methods described herein further include: (d) performing a plurality of mammalian cell culture runs in the perfusion bioreactor having the DO probe positioned at the selected fixed orientation, where each of the plurality of mammalian cell culture runs has a different measured DO level as measured by the DO probe; (e) detecting one or more of viable cell density, lactate production, and recombinant protein production by cells in each mammalian cell culture run; and (f) selecting a DO level for additional mammalian cell culture runs in the perfusion bioreactor based on one or more of the detected viable cell density, lactate production, and recombinant protein production in step (e) for each mammalian cell culture run.

Some embodiments of any of the methods described herein further include culturing a mammalian cell in a perfusion bioreactor having a DO probe at the selected fixed orientation in a liquid culture medium under conditions that result in the selected DO level.

In some embodiments of any of the methods described herein, the DO probe is a rod-shaped DO probe. In some embodiments of any of the methods described herein, the DO probe comprises an optical sensor. In some embodiments of any of the methods described herein, the DO probe has a base and a tip, wherein the optical sensor is located at the tip-end of the DO probe.

In some embodiments of any of the methods described herein, the fixed orientation is identified by a fixed angle of the DO probe. In some embodiments of any of the methods described herein, the plurality of different fixed orientations is a plurality of different fixed angles between about 0 degrees and about 180 degrees. In some embodiments of any of the methods described herein, the plurality of fixed angles are between about 0 degree and about 90 degrees. In some embodiments of any of the methods described herein, the plurality of fixed angles are between about 90 degrees and about 180 degrees.

In some embodiments of any of the methods described herein, the tip of the DO probe is tilted or slanted. In some embodiments of any of the methods described herein, each of the plurality of different fixed orientations is identified by a fixed angle of the DO probe and degree of rotation of the DO probe along the longitudinal axis of the DO probe. In some embodiments of any of the methods described herein, each of the plurality of different fixed orientations have a shared fixed angle of the DO probe and different degrees of rotation of the DO probe along the longitudinal axis of the DO probe. In some embodiments of any of the methods described herein, the plurality of different fixed orientations are a plurality of different degrees of rotation of the DO probe along the longitudinal axis of the DO probe in a plurality of different fixed angles. In some embodiments of any of the methods described herein, the different degrees of rotations are between about 0 degree and about 360 degrees. In some embodiments of any of the methods described herein, the different degrees of rotation are between about 0 degree and about 180 degrees. In some embodiments of any of the methods described herein, the different degrees of rotation are between about 180 degree and about 360 degrees.

In some embodiments of any of the methods described herein, the selected fixed orientation of the DO probe reduces accumulation of air bubbles on or near the sensor of the DO probe.

In some embodiments of any of the methods described herein, one or more of volumetric input power, culturing temperature, and culture volume are adjusted to achieve the selected DO level. In some embodiments of any of the methods described herein, the internal volume of the perfusion bioreactor is about 5 L to about 1000 L. In some embodiments of any of the methods described herein, the internal volume of the perfusion bioreactor is about 100 L to about 500 L.

In some embodiments of any of the methods described herein, the mammalian cell is a mammalian cell line. In some embodiments of any of the methods described herein, the mammalian cell line is a Chinese Hamster Ovarian (CHO) cell line.

Also provided herein are methods of culturing a mammalian cell in a perfusion bioreactor that include: (a) providing a perfusion bioreactor having a DO probe at a fixed orientation with a fixed angle of about 60-70 degrees and about a 0-degree to 10-degree rotation and containing a mammalian cell in a liquid culture medium; and (b) culturing the mammalian cell using the perfusion bioreactor under conditions sufficient to achieve a DO level of about 20% to about 40%, wherein the perfusion bioreactor has an internal volume of about 5 L to about 1000 L.

In some embodiments of any of the methods described herein, the conditions sufficient to achieve a DO level of about 20% to about 40% comprises a volumetric input power of about 30 W/m³ to about 90 W/m³. In some embodiments of any of the methods described herein, the conditions sufficient to achieve a DO level of about 20% to about 40% comprises a volumetric input power of about 36 W/m³ to about 65 W/m³.

In some embodiments of any of the methods described herein, the internal volume of the perfusion bioreactor is about 5 L to about 100 L. In some embodiments of any of the methods described herein, the internal volume of the perfusion bioreactor is about 5 L to about 15 L. In some embodiments of any of the methods described herein, the internal volume of the perfusion bioreactor is about 100 L to about 500 L.

In some embodiments of any of the methods described herein, the volume of the liquid culture medium is about 5 L to about 100 L. In some embodiments of any of the methods described herein, the volume of the liquid culture medium is about 5 L to about 15 L. In some embodiments of any of the methods described herein, the volume of the liquid culture medium is about 100 L to about 500 L.

In some embodiments of any of the methods described herein, the mammalian cell is a mammalian cell line. In some embodiments of any of the methods described herein, the mammalian cell line is a Chinese Hamster Ovarian (CHO) cell line.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color.

FIG. 1 is a schematic Illustration of a perfusion bioreactor equipped with a dissolved oxygen (DO) probe, an optical density (OD) probe, a pH probe, and a CO₂ probe.

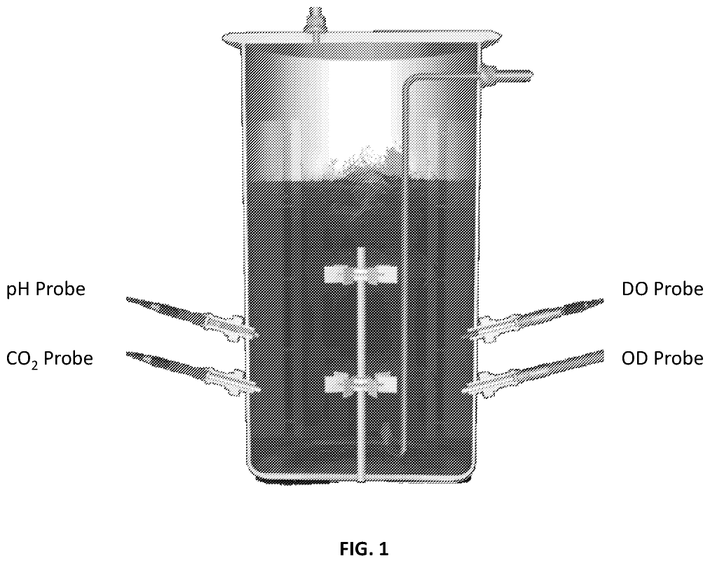

FIG. 2 shows an example of a commercially available DO probe InPro6860i (Mettler Toledo).

FIGS. 3A-3E are schematic illustrations of fixed angles of a DO probe in a perfusion bioreactor. The fixed angles of the DO probe are depicted relative to a vertical axis that is in parallel to the side wall of a bioreactor that has a cylindrical shape.

FIGS. 4A-4B are schematic illustrations of different orientations of a DO probe having a fixed angle of about 90 degrees and different levels of rotation along its longitudinal axis. FIG. 4A shows the DO probe having a fixed angle of about 90 degrees and a 0-degree rotation along its longitudinal axis and FIG. 4B shows the DO probe having a fixed angle of about 90 degree and a 180-degree rotation along its longitudinal axis. The rotation of the DO probe is depicted in a direction along the longitudinal axis of the DO probe.

FIGS. 5A-5D show the impact of high DO level on culture performance at pilot scales. FIG. 5A shows the viable cell density (VCD). FIG. 5B shows cell viability. FIG. 5C shows the lactate concentrations. FIG. 5D shows the glutamine concentration of five pilot scale batches. Runs 1-3 were the first pilot scale batches and establish a baseline while Runs 4-5 exhibited dysfunctional behavior and were terminated early.

FIGS. 6A-6D show the results of benchtop cell culture under various DO levels. FIG. 6A shows the viable cell density (VCD). FIG. 6B shows cell viability. FIG. 6C shows the lactate concentrations. FIG. 6D shows the glutamine concentration of benchtop bioreactors with DO setpoints ranging from 30 to 90%.

FIGS. 7A-7B show the results of process scaling-up with improved DO measuring and mixing. FIG. 7A shows cell viability. FIG. 7B show the lactate concentrations of pilot scale Runs 1-7. Runs 1-5 correspond to the previously visualized batches from FIGS. 5A-5D. Runs 6-7 were run with the decreased DO setpoint and improved mixing environment.

### DETAILED DESCRIPTION

The disclosure relates to methods for culturing a mammalian cell and selecting a fixed orientation of a DO probe in a perfusion bioreactor to allow the accurate measurement of the DO level in a culture (e.g., a mammalian cell culture). The present invention is based on the discovery that the orientation of the DO probe in a bioreactor (e.g., a perfusion bioreactor) is critical for the accurate measurement of DO level. A proper orientation (e.g., proper fixed angle) of a DO probe in a bioreactor (e.g., a perfusion bioreactor) can eliminate the detection of falsely increased DO levels caused by, e.g., the accumulation of air bubbles on or near the sensor of the DO probe in the bioreactor, and/or falsely decreased DO levels. A carefully selected fixed orientation of a DO probe in a pilot scale perfusion process also allows the identification of more robust operating ranges and a more robust scale-up process for the production of proteins such as antibodies.

For example, once a fixed orientation of the DO probe is selected, additional mammalian cell culture runs can be performed to determine the preferred DO level to be used in mammalian cell cultures, and culture conditions can be modified to achieve the preferred (selected) DO level in the bioreactor (e.g., perfusion bioreactor).

The disclosure also relates to methods of adjusting one or more culturing conditions based on the DO measurement from the selected, optimal fixed orientation of the DO probe. The culturing conditions include, for example, the DO setpoint and the volumetric input power for the bioreactor (e.g., perfusion bioreactor).

### Definitions

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell culture medium, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

“Culture” or “cell culture” refers to the maintenance, growth and/or differentiation of cells (e.g., mammalian cells) in an in vitro environment. Cell culture can refer in some embodiments to batch cell culture, fed-batch cell culture, or perfusion cell culture. “Cell culture media,” “culture media” (singular “medium” in each case), “supplement” and “media supplement” refer to nutritive compositions that cultivate cell cultures.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

The term “recombinant protein,” as used herein, refers to any protein or biologically active portion thereof (for example, a portion that retains biological activity of the full protein) that is not a reporter or marker gene (for example, a green fluorescent protein) expressed from recombinant genetic material encoding amino acids, including peptides, polypeptides, proteins, oligoproteins and/or fusion proteins. A recombinant protein product may include a therapeutic, prophylactic, or diagnostic product.

The term “production of protein” includes techniques used to grow cells, e.g., recombinant cells, in culture and to obtain a protein of interest produced by the cultured cells in an appropriate form for use. The manufacturing process can include various steps, including, but not limited to one or more of the following: inserting of a gene of interest into a host cell to create an engineered host cell, culturing the host cell to expand the number of cells, inducing expression of the protein of interest by the host cell, screening for host cells expressing the protein of interest, harvesting the protein of interest, e.g., by separating the protein of interest from the cultured cells and cell culture medium, and/or purifying the protein of interest. The protein of interest can be an endogenous protein expressed by the native cell, or a recombinant heterologous protein encoded in an expression vector inserted into the cell (either transiently or stably).

In one aspect, provided herein is a method of culturing a mammalian cell in a perfusion bioreactor, wherein the method comprises: (a) placing a dissolved oxygen (DO) probe in the perfusion bioreactor comprising a liquid at a plurality of different fixed orientations; (b) measuring the DO level using the DO probe at each of the plurality of different fixed orientations; (c) selecting a fixed orientation for the DO probe in the bioreactor based on the measured DO levels in step (b), wherein the selected fixed orientation does not demonstrate measurement of falsely increased DO levels as determined by analysis of the measured DO levels in step (b); and (d) culturing a mammalian cell in a liquid culture medium in the perfusion bioreactor having the DO probe positioned at the selected fixed orientation.

Also provided herein is a method of culturing a cell in a perfusion bioreactor, wherein the method comprises: (a) placing a dissolved oxygen (DO) probe in the perfusion bioreactor comprising a liquid at a plurality of different fixed orientations; (b) measuring the DO level using the DO probe at each of the plurality of different fixed orientations; (c) selecting a fixed orientation for the DO probe in the bioreactor based on the measured DO levels in step (b), wherein the selected fixed orientation does not demonstrate measurement of falsely increased DO levels as determined by analysis of the measured DO levels in step (b); and (d) culturing a cell in a liquid culture medium in the perfusion bioreactor having the DO probe positioned at the selected fixed orientation.

Also provided herein is a method of selecting a fixed orientation for a dissolved oxygen (DO) probe in a perfusion bioreactor, the method comprising: (a) placing the DO probe in the perfusion bioreactor comprising a liquid at a plurality of different fixed orientations; (b) measuring the DO level using the DO probe at each of the plurality of different fixed orientations; and (c) selecting a fixed orientation for the DO probe in the bioreactor based on the measured DO levels in step (b), wherein the selected fixed orientation does not demonstrate measurement of falsely increased DO levels as determined by analysis of the measured DO levels in step (b).

In some embodiments, the method further includes: (d) performing a plurality of mammalian cell culture runs in the perfusion bioreactor having the DO probe positioned at the selected fixed orientation, where each of the plurality of mammalian cell culture runs has a different measured DO level as measured by the DO probe; (e) detecting one or more of viable cell density, lactate production, and recombinant protein production by cells in each mammalian cell culture run; and (f) selecting a DO level for additional mammalian cell culture runs in the perfusion bioreactor based on one or more of the detected viable cell density, lactate production, and recombinant protein production in step (e) for each mammalian cell culture run.

In some embodiments, the method further includes culturing a mammalian cell in a perfusion bioreactor having a DO probe at the selected fixed orientation in a liquid culture medium under conditions that result in the selected DO level.

In some embodiments, the method further includes: (d) performing a plurality of cell culture runs in the perfusion bioreactor having the DO probe positioned at the selected fixed orientation, where each of the plurality of cell culture runs has a different measured DO level as measured by the DO probe; (e) detecting one or more of viable cell density, lactate production, and recombinant protein production by cells in each cell culture run; and (f) selecting a DO level for additional cell culture runs in the perfusion bioreactor based on one or more of the detected viable cell density, lactate production, and recombinant protein production in step (e) for each cell culture run.

In some embodiments, the method further includes culturing a cell in a perfusion bioreactor having a DO probe at the selected fixed orientation in a liquid culture medium under conditions that result in the selected DO level.

Any suitable DO probes can be used in the methods described herein. Examples of commercially available DO probes are described in the Examples herein. In some embodiments, the DO probe is a rod-shaped DO probe.

Examples of dissolved oxygen sensors include galvanic dissolved oxygen sensors, polarographic dissolved oxygen sensors, and optical dissolved oxygen sensors. Each type of dissolved oxygen sensor has a slightly different working principle. Both galvanic DO sensors and polarographic DO sensors are types of electrochemical dissolved oxygen sensors. In an electrochemical DO sensor, dissolved oxygen diffuses from the sample across an oxygen permeable membrane and into the sensor. Once inside the sensor, the oxygen undergoes a chemical reduction reaction, which produces an electrical signal. This signal can be read by a dissolved oxygen instrument.

The difference between a galvanic DO sensor and a polarographic DO sensor is that a polarographic DO sensor requires a constant voltage to be applied to it. It must be polarized. By contrast, a galvanic DO sensor is self-polarizing due to the material properties of the anode (zinc or lead) and cathode (silver). This means that while galvanic DO sensors can be used immediately after calibration, polarographic sensors require a 5-15 minute warm up time.

An optical dissolved oxygen sensor does not have an anode or cathode, and oxygen is not reduced during measurement. Instead, the sensor cap contains a luminescent dye, which glows red when exposed to blue light. Oxygen interferes with the luminescent properties of the dye, an effect called “quenching.” A photodiode compares the “quenched” luminescence to a reference reading, allowing the calculation of dissolved oxygen concentration in water.

In some embodiments, the DO probe used in the method described herein includes an optical sensor. In some embodiments, the DO probe has a base and a tip, wherein the optical sensor is located at the tip-end of the probe.

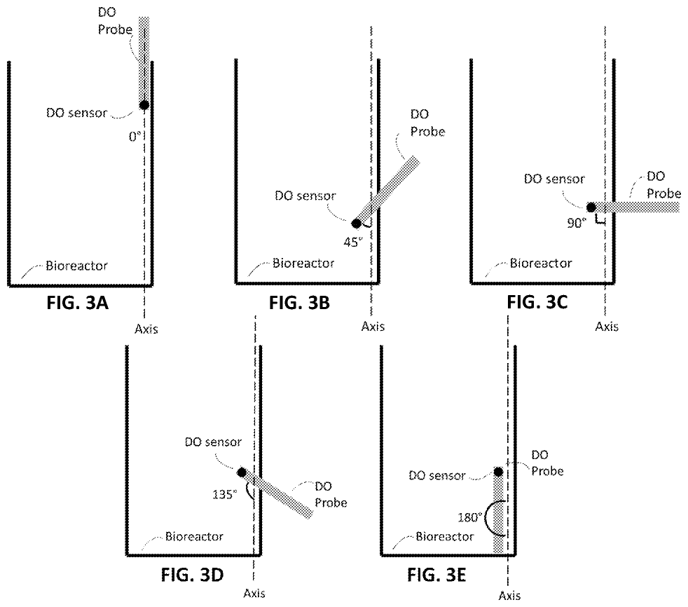

In some embodiments, the fixed orientation of the DO probe is identified by a fixed angle of the DO probe. As used herein, a “fixed angle” is an angle between the portion of the longitudinal axis of a DO probe (e.g., a rod-shaped DO probe) that is exposed to the interior space of a bioreactor, and a vertical axis essentially in parallel with the side wall of the bioreactor (e.g., a cylindrical bioreactor), where a non-sensing portion (e.g., end) of the DO probe is attached to a side wall or the bottom wall of the bioreactor. The directionality of the fixed angles described herein are illustrated in FIGS. 3A-3E. In some embodiments, the plurality of different fixed orientations is a plurality of different fixed angles between about 0 degree and about 180 degrees. In some embodiments, the plurality of different fixed orientations is a plurality of different fixed angles between about 0 degree and about 90 degrees. In some embodiments, the plurality of different fixed orientations is a plurality of different fixed angles between about 90 degree and about 180 degrees. In some embodiments, the plurality of different fixed angels is between about 0 degree and 180 degrees, about 0 degree and about 175 degrees, about 0 degree and about 170 degrees, about 0 degree and about 165 degrees, about 0 degree and about 160 degrees, about 0 degree and about 155 degrees, about 0 degree and about 150 degrees, about 0 degree and about 145 degrees, about 0 degree and about 140 degrees, about 0 degree and about 135 degrees, about 0 degree and about 130 degrees, about 0 degree and about 125 degrees, about 0 degree and about 120 degrees, about 0 degree and about 115 degrees, about 0 degree and about 110 degrees, about 0 degree and about 105 degrees, about 0 degree and about 100 degrees, about 0 degree to about 95 degrees, about 0 degree to about 90 degrees, about 0 degree to about 85 degrees, about 0 degree to about 80 degrees, about 0 degree to about 75 degrees, about 0 degree to about 70 degrees, about 0 degree to about 65 degrees, about 0 degree to about 60 degrees, about 0 degree to about 55 degrees, about 0 degree to about 50 degrees, about 0 degree to about 45 degrees, about 0 degree to about 40 degrees, about 0 degree to about 35 degrees, about 0 degree to about 30 degrees, about 0 degree to about 25 degrees, between about 5 degrees and 180 degrees, about 5 degrees and about 175 degrees, about 5 degrees and about 170 degrees, about 5 degrees and about 165 degrees, about 5 degrees and about 160 degrees, about 5 degrees and about 155 degrees, about 5 degrees and about 150 degrees, about 5 degrees and about 145 degrees, about 5 degrees and about 140 degrees, about 5 degrees and about 135 degrees, about 5 degrees and about 130 degrees, about 5 degrees and about 125 degrees, about 5 degrees and about 120 degrees, about 5 degrees and about 115 degrees, about 5 degrees and about 110 degrees, about 5 degrees and about 105 degrees, about 5 degrees and about 100 degrees, about 5 degrees to about 95 degrees, about 5 degrees to about 90 degrees, about 5 degrees to about 85 degrees, about 5 degrees to about 80 degrees, about 5 degrees to about 75 degrees, about 5 degrees to about 70 degrees, about 5 degrees to about 65 degrees, about 5 degrees to about 60 degrees, about 5 degrees to about 55 degrees, about 5 degrees to about 50 degrees, about 5 degrees to about 45 degrees, about 5 degrees to about 40 degrees, about 5 degrees to about 35 degrees, about 5 degrees to about 30 degrees, between about 10 degrees and 180 degrees, about 10 degrees and about 175 degrees, about 10 degrees and about 170 degrees, about 10 degrees and about 165 degrees, about 10 degrees and about 160 degrees, about 10 degrees and about 155 degrees, about 10 degrees and about 150 degrees, about 10 degrees and about 145 degrees, about 10 degrees and about 140 degrees, about 10 degrees and about 135 degrees, about 10 degrees and about 130 degrees, about 10 degrees and about 125 degrees, about 10 degrees and about 120 degrees, about 10 degrees and about 115 degrees, about 10 degrees and about 110 degrees, about 10 degrees and about 105 degrees, about 10 degrees and about 100 degrees, about 10 degrees to about 95 degrees, about 10 degrees to about 90 degrees, about 10 degrees to about 85 degrees, about 10 degrees to about 80 degrees, about 10 degrees to about 75 degrees, about 10 degrees to about 70 degrees, about 10 degrees to about 65 degrees, about 10 degrees to about 60 degrees, about 10 degrees to about 55 degrees, about 10 degrees to about 50 degrees, about 10 degrees to about 45 degrees, about 10 degrees to about 40 degrees, about 10 degrees to about 35 degrees, between about 15 degrees and 180 degrees, about 15 degrees and about 175 degrees, about 15 degrees and about 170 degrees, about 15 degrees and about 165 degrees, about 15 degrees and about 160 degrees, about 15 degrees and about 155 degrees, about 15 degrees and about 150 degrees, about 15 degrees and about 145 degrees, about 15 degrees and about 140 degrees, about 15 degrees and about 135 degrees, about 15 degrees and about 130 degrees, about 15 degrees and about 125 degrees, about 15 degrees and about 120 degrees, about 15 degrees and about 115 degrees, about 15 degrees and about 110 degrees, about 15 degrees and about 105 degrees, about 15 degrees and about 100 degrees, about 15 degrees to about 95 degrees, about 15 degrees to about 90 degrees, about 15 degrees to about 85 degrees, about 15 degrees to about 80 degrees, about 15 degrees to about 75 degrees, about 15 degrees to about 70 degrees, about 15 degrees to about 65 degrees, about 15 degrees to about 60 degrees, about 15 degrees to about 55 degrees, about 15 degrees to about 50 degrees, about 15 degrees to about 45 degrees, about 15 degrees to about 40 degrees, between about 20 degrees and 180 degrees, about 20 degrees and about 175 degrees, about 20 degrees and about 170 degrees, about 20 degrees and about 165 degrees, about 20 degrees and about 160 degrees, about 20 degrees and about 155 degrees, about 20 degrees and about 150 degrees, about 20 degrees and about 145 degrees, about 20 degrees and about 140 degrees, about 20 degrees and about 135 degrees, about 20 degrees and about 130 degrees, about 20 degrees and about 125 degrees, about 20 degrees and about 120 degrees, about 20 degrees and about 115 degrees, about 20 degrees and about 110 degrees, about 20 degrees and about 105 degrees, about 20 degrees and about 100 degrees, about 20 degrees to about 95 degrees, about 20 degrees to about 90 degrees, about 20 degrees to about 85 degrees, about 20 degrees to about 80 degrees, about 20 degrees to about 75 degrees, about 20 degrees to about 70 degrees, about 20 degrees to about 65 degrees, about 20 degrees to about 60 degrees, about 20 degrees to about 55 degrees, about 20 degrees to about 50 degrees, about 20 degrees to about 45 degrees, between about 25 degrees and 180 degrees, about 25 degrees and about 175 degrees, about 25 degrees and about 170 degrees, about 25 degrees and about 165 degrees, about 25 degrees and about 160 degrees, about 25 degrees and about 155 degrees, about 25 degrees and about 150 degrees, about 25 degrees and about 145 degrees, about 25 degrees and about 140 degrees, about 25 degrees and about 135 degrees, about 25 degrees and about 130 degrees, about 25 degrees and about 125 degrees, about 25 degrees and about 120 degrees, about 25 degrees and about 115 degrees, about 25 degrees and about 110 degrees, about 25 degrees and about 105 degrees, about 25 degrees and about 100 degrees, about 25 degrees to about 95 degrees, about 25 degrees to about 90 degrees, about 25 degrees to about 85 degrees, about 25 degrees to about 80 degrees, about 25 degrees to about 75 degrees, about 25 degrees to about 70 degrees, about 25 degrees to about 65 degrees, about 25 degrees to about 60 degrees, about 25 degrees to about 55 degrees, about 25 degrees to about 50 degrees, between about 30 degrees and 180 degrees, about 30 degrees and about 175 degrees, about 30 degrees and about 170 degrees, about 30 degrees and about 165 degrees, about 30 degrees and about 160 degrees, about 30 degrees and about 155 degrees, about 30 degrees and about 150 degrees, about 30 degrees and about 145 degrees, about 30 degrees and about 140 degrees, about 30 degrees and about 135 degrees, about 30 degrees and about 130 degrees, about 30 degrees and about 125 degrees, about 30 degrees and about 120 degrees, about 30 degrees and about 115 degrees, about 30 degrees and about 110 degrees, about 30 degrees and about 105 degrees, about 30 degrees and about 100 degrees, about 30 degrees to about 95 degrees, about 30 degrees to about 90 degrees, about 30 degrees to about 85 degrees, about 30 degrees to about 80 degrees, about 30 degrees to about 75 degrees, about 30 degrees to about 70 degrees, about 30 degrees to about 65 degrees, about 30 degrees to about 60 degrees, about 30 degrees to about 55 degrees, between about 35 degrees and 180 degrees, about 35 degrees and about 175 degrees, about 35 degrees and about 170 degrees, about 35 degrees and about 165 degrees, about 35 degrees and about 160 degrees, about 35 degrees and about 155 degrees, about 35 degrees and about 150 degrees, about 35 degrees and about 145 degrees, about 35 degrees and about 140 degrees, about 35 degrees and about 135 degrees, about 35 degrees and about 130 degrees, about 35 degrees and about 125 degrees, about 35 degrees and about 120 degrees, about 35 degrees and about 115 degrees, about 35 degrees and about 110 degrees, about 35 degrees and about 105 degrees, about 35 degrees and about 100 degrees, about 35 degrees to about 95 degrees, about 35 degrees to about 90 degrees, about 35 degrees to about 85 degrees, about 35 degrees to about 80 degrees, about 35 degrees to about 75 degrees, about 35 degrees to about 70 degrees, about 35 degrees to about 65 degrees, about 35 degrees to about 60 degrees, between about 40 degrees and 180 degrees, about 40 degrees and about 175 degrees, about 40 degrees and about 170 degrees, about 40 degrees and about 165 degrees, about 40 degrees and about 160 degrees, about 40 degrees and about 155 degrees, about 40 degrees and about 150 degrees, about 40 degrees and about 145 degrees, about 40 degrees and about 140 degrees, about 40 degrees and about 135 degrees, about 40 degrees and about 130 degrees, about 40 degrees and about 125 degrees, about 40 degrees and about 120 degrees, about 40 degrees and about 115 degrees, about 40 degrees and about 110 degrees, about 40 degrees and about 105 degrees, about 40 degrees and about 100 degrees, about 40 degrees to about 95 degrees, about 40 degrees to about 90 degrees, about 40 degrees to about 85 degrees, about 40 degrees to about 80 degrees, about 40 degrees to about 75 degrees, about 40 degrees to about 70 degrees, about 40 degrees to about 65 degrees, between about 45 degrees and 180 degrees, about 45 degrees and about 175 degrees, about 45 degrees and about 170 degrees, about 45 degrees and about 165 degrees, about 45 degrees and about 160 degrees, about 45 degrees and about 155 degrees, about 45 degrees and about 150 degrees, about 45 degrees and about 145 degrees, about 45 degrees and about 140 degrees, about 45 degrees and about 135 degrees, about 45 degrees and about 130 degrees, about 45 degrees and about 125 degrees, about 45 degrees and about 120 degrees, about 45 degrees and about 115 degrees, about 45 degrees and about 110 degrees, about 45 degrees and about 105 degrees, about 45 degrees and about 100 degrees, about 45 degrees to about 95 degrees, about 45 degrees to about 90 degrees, about 45 degrees to about 85 degrees, about 45 degrees to about 80 degrees, about 45 degrees to about 75 degrees, about 45 degrees to about 70 degrees, between about 50 degrees and 180 degrees, about 50 degrees and about 175 degrees, about 50 degrees and about 170 degrees, about 50 degrees and about 165 degrees, about 50 degrees and about 160 degrees, about 50 degrees and about 155 degrees, about 50 degrees and about 150 degrees, about 50 degrees and about 145 degrees, about 50 degrees and about 140 degrees, about 50 degrees and about 135 degrees, about 50 degrees and about 130 degrees, about 50 degrees and about 125 degrees, about 50 degrees and about 120 degrees, about 50 degrees and about 115 degrees, about 50 degrees and about 110 degrees, about 50 degrees and about 105 degrees, about 50 degrees and about 100 degrees, about 50 degrees to about 95 degrees, about 50 degrees to about 90 degrees, about 50 degrees to about 85 degrees, about 50 degrees to about 80 degrees, about 50 degrees to about 75 degrees, between about 55 degrees and 180 degrees, about 55 degrees and about 175 degrees, about 55 degrees and about 170 degrees, about 55 degrees and about 165 degrees, about 55 degrees and about 160 degrees, about 55 degrees and about 155 degrees, about 55 degrees and about 150 degrees, about 55 degrees and about 145 degrees, about 55 degrees and about 140 degrees, about 55 degrees and about 135 degrees, about 55 degrees and about 130 degrees, about 55 degrees and about 125 degrees, about 55 degrees and about 120 degrees, about 55 degrees and about 115 degrees, about 55 degrees and about 110 degrees, about 55 degrees and about 105 degrees, about 55 degrees and about 100 degrees, about 55 degrees to about 95 degrees, about 55 degrees to about 90 degrees, about 55 degrees to about 85 degrees, about 55 degrees to about 80 degrees, between about 60 degrees and 180 degrees, about 60 degrees and about 175 degrees, about 60 degrees and about 170 degrees, about 60 degrees and about 165 degrees, about 60 degrees and about 160 degrees, about 60 degrees and about 155 degrees, about 60 degrees and about 150 degrees, about 60 degrees and about 145 degrees, about 60 degrees and about 140 degrees, about 60 degrees and about 135 degrees, about 60 degrees and about 130 degrees, about 60 degrees and about 125 degrees, about 60 degrees and about 120 degrees, about 60 degrees and about 115 degrees, about 60 degrees and about 110 degrees, about 60 degrees and about 105 degrees, about 60 degrees and about 100 degrees, about 60 degrees to about 95 degrees, about 60 degrees to about 90 degrees, about 60 degrees to about 85 degrees, between about 65 degrees and 180 degrees, about 65 degrees and about 175 degrees, about 65 degrees and about 170 degrees, about 65 degrees and about 165 degrees, about 65 degrees and about 160 degrees, about 65 degrees and about 155 degrees, about 65 degrees and about 150 degrees, about 65 degrees and about 145 degrees, about 65 degrees and about 140 degrees, about 65 degrees and about 135 degrees, about 65 degrees and about 130 degrees, about 65 degrees and about 125 degrees, about 65 degrees and about 120 degrees, about 65 degrees and about 115 degrees, about 65 degrees and about 110 degrees, about 65 degrees and about 105 degrees, about 65 degrees and about 100 degrees, about 65 degrees to about 95 degrees, about 65 degrees to about 90 degrees, between about 70 degrees and 180 degrees, about 70 degrees and about 175 degrees, about 70 degrees and about 170 degrees, about 70 degrees and about 165 degrees, about 70 degrees and about 160 degrees, about 70 degrees and about 155 degrees, about 70 degrees and about 150 degrees, about 70 degrees and about 145 degrees, about 70 degrees and about 140 degrees, about 70 degrees and about 135 degrees, about 70 degrees and about 130 degrees, about 70 degrees and about 125 degrees, about 70 degrees and about 120 degrees, about 70 degrees and about 115 degrees, about 70 degrees and about 110 degrees, about 70 degrees and about 105 degrees, about 70 degrees and about 100 degrees, about 70 degrees to about 95 degrees, between about 75 degrees and 180 degrees, about 75 degrees and about 175 degrees, about 75 degrees and about 170 degrees, about 75 degrees and about 165 degrees, about 75 degrees and about 160 degrees, about 75 degrees and about 155 degrees, about 75 degrees and about 150 degrees, about 75 degrees and about 145 degrees, about 75 degrees and about 140 degrees, about 75 degrees and about 135 degrees, about 75 degrees and about 130 degrees, about 75 degrees and about 125 degrees, about 75 degrees and about 120 degrees, about 75 degrees and about 115 degrees, about 75 degrees and about 110 degrees, about 75 degrees and about 105 degrees, about 75 degrees and about 100 degrees, between about 80 degrees and 180 degrees, about 80 degrees and about 175 degrees, about 80 degrees and about 170 degrees, about 80 degrees and about 165 degrees, about 80 degrees and about 160 degrees, about 80 degrees and about 155 degrees, about 80 degrees and about 150 degrees, about 80 degrees and about 145 degrees, about 80 degrees and about 140 degrees, about 80 degrees and about 135 degrees, about 80 degrees and about 130 degrees, about 80 degrees and about 125 degrees, about 80 degrees and about 120 degrees, about 80 degrees and about 115 degrees, about 80 degrees and about 110 degrees, about 80 degrees and about 105 degrees, between about 85 degrees and 180 degrees, about 85 degrees and about 175 degrees, about 85 degrees and about 170 degrees, about 85 degrees and about 165 degrees, about 85 degrees and about 160 degrees, about 85 degrees and about 155 degrees, about 85 degrees and about 150 degrees, about 85 degrees and about 145 degrees, about 85 degrees and about 140 degrees, about 85 degrees and about 135 degrees, about 85 degrees and about 130 degrees, about 85 degrees and about 125 degrees, about 85 degrees and about 120 degrees, about 85 degrees and about 115 degrees, about 85 degrees and about 110 degrees, between about 90 degrees and 180 degrees, about 90 degrees and about 175 degrees, about 90 degrees and about 170 degrees, about 90 degrees and about 165 degrees, about 90 degrees and about 160 degrees, about 90 degrees and about 155 degrees, about 90 degrees and about 150 degrees, about 90 degrees and about 145 degrees, about 90 degrees and about 140 degrees, about 90 degrees and about 135 degrees, about 90 degrees and about 130 degrees, about 90 degrees and about 125 degrees, about 90 degrees and about 120 degrees, about 90 degrees and about 115 degrees, between about 95 degrees and 180 degrees, about 95 degrees and about 175 degrees, about 95 degrees and about 170 degrees, about 95 degrees and about 165 degrees, about 95 degrees and about 160 degrees, about 95 degrees and about 155 degrees, about 95 degrees and about 150 degrees, about 95 degrees and about 145 degrees, about 95 degrees and about 140 degrees, about 95 degrees and about 135 degrees, about 95 degrees and about 130 degrees, about 95 degrees and about 125 degrees, about 95 degrees and about 120 degrees, between about 100 degrees and 180 degrees, about 100 degrees and about 175 degrees, about 100 degrees and about 170 degrees, about 100 degrees and about 165 degrees, about 100 degrees and about 160 degrees, about 100 degrees and about 155 degrees, about 100 degrees and about 150 degrees, about 100 degrees and about 145 degrees, about 100 degrees and about 140 degrees, about 100 degrees and about 135 degrees, about 100 degrees and about 130 degrees, about 100 degrees and about 125 degrees, between about 105 degrees and 180 degrees, about 105 degrees and about 175 degrees, about 105 degrees and about 170 degrees, about 105 degrees and about 165 degrees, about 105 degrees and about 160 degrees, about 105 degrees and about 155 degrees, about 105 degrees and about 150 degrees, about 105 degrees and about 145 degrees, about 105 degrees and about 140 degrees, about 105 degrees and about 135 degrees, about 105 degrees and about 130 degrees, between about 110 degrees and 180 degrees, about 110 degrees and about 175 degrees, about 110 degrees and about 170 degrees, about 110 degrees and about 165 degrees, about 110 degrees and about 160 degrees, about 110 degrees and about 155 degrees, about 110 degrees and about 150 degrees, about 110 degrees and about 145 degrees, about 110 degrees and about 140 degrees, about 110 degrees and about 135 degrees, between about 115 degrees and 180 degrees, about 115 degrees and about 175 degrees, about 115 degrees and about 170 degrees, about 115 degrees and about 165 degrees, about 115 degrees and about 160 degrees, about 115 degrees and about 155 degrees, about 115 degrees and about 150 degrees, about 115 degrees and about 145 degrees, about 115 degrees and about 140 degrees, between about 120 degrees and 180 degrees, about 120 degrees and about 175 degrees, about 120 degrees and about 170 degrees, about 120 degrees and about 165 degrees, about 120 degrees and about 160 degrees, about 120 degrees and about 155 degrees, about 120 degrees and about 150 degrees, about 120 degrees and about 145 degrees, between about 125 degrees and 180 degrees, about 125 degrees and about 175 degrees, about 125 degrees and about 170 degrees, about 125 degrees and about 165 degrees, about 125 degrees and about 160 degrees, about 125 degrees and about 155 degrees, about 125 degrees and about 150 degrees, between about 130 degrees and 180 degrees, about 130 degrees and about 175 degrees, about 130 degrees and about 170 degrees, about 130 degrees and about 165 degrees, about 130 degrees and about 160 degrees, about 130 degrees and about 155 degrees, between about 135 degrees and 180 degrees, about 135 degrees and about 175 degrees, about 135 degrees and about 170 degrees, about 135 degrees and about 165 degrees, about 135 degrees and about 160 degrees, between about 140 degrees and 180 degrees, about 140 degrees and about 175 degrees, about 140 degrees and about 170 degrees, about 140 degrees and about 165 degrees, between about 145 degrees and 180 degrees, about 145 degrees and about 175 degrees, about 145 degrees and about 170 degrees, between about 150 degrees and 180 degrees, about 150 degrees and about 175 degrees, or between about 155 degrees and about 180 degrees.

In some embodiments, the plurality of different fixed orientations is a plurality of different fixed angles selected from the group of about 0, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, and 180 degrees (e.g., as illustrated in FIGS. 3A-3E).

In some embodiments, the DO probe has a tilted or slanted sensing tip or end. In some embodiments, the titled or slanted tip includes a DO sensor window. The tilted or slanted tip of the DO probe provides anisotropy when it comes to the rotation of the probe within the bioreactor.

In some embodiments, the sensing tip of the DO probe is tilted or slanted about 5 degrees, about 10 degrees, about 15 degrees, about 20 degrees, about 25, degrees, about 30 degrees, about 35 degrees, about 40 degrees, about 45 degrees, about 50 degrees, about 55 degrees, about 60 degrees, about 65 degrees, about 70 degrees, about 75 degrees, or about 80 degrees. Any other suitable tilted- or slanted-tip DO probe can also be used in any of the methods described herein.

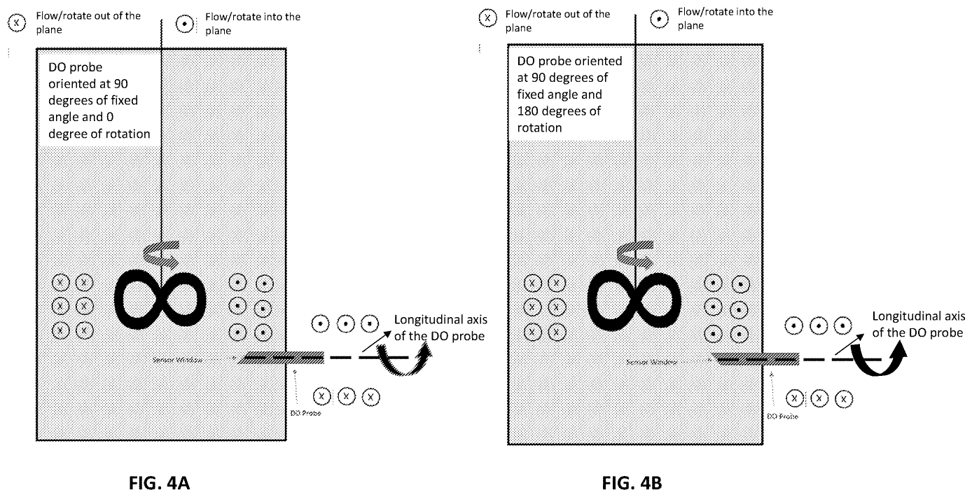

In some embodiments, the plurality of different fixed orientations of the DO probe can be the DO probe positioned at the same fixed angle (e.g., any of the exemplary fixed angles described herein, e.g., about 60-70 degrees), but having different degrees of rotation (e.g., 0 degrees to about 360 degrees) about its longitudinal axis. Different exemplary degrees of rotation of a DO probe positioned at the same fixed angle is illustrated in FIGS. 4A-4B. In such embodiments, a degree of rotation of 0 degrees represents when the tilted- or slanted-tip of the DO probe is facing the operator and the rotation along the longitudinal axis of the DO probe occurs in the direction as shown in FIGS. 4A-4B. In some embodiments, the plurality of different fixed orientations is a plurality of degrees of rotation of a DO probe along its longitudinal axis (e.g., between 0 degree and about 360 degrees) with the DO probe positioned at a plurality of different fixed angles (e.g., two or more of any of the exemplary fixed angles described herein or any of the exemplary subranges of fixed angles described herein).

In some embodiments, the plurality of different fixed orientations is a plurality of different degrees of rotation of the DO probe (along its longitudinal axis), when the DO probe is positioned at the same fixed angle (e.g., any of the exemplary fixed angles described herein), is between about 0 degree and about 360 degrees of rotation, between about 0 degree and about 180 degrees of rotation, or between about 180 degree and about 360 degrees or rotation. As would be appreciated by those in the art, a rotation of 360 degrees is the same position as a rotation of 0 degree, when the DO probe is positioned at the same fixed angle.

In some embodiments, the plurality of different fixed orientations is a plurality of different degrees of rotation of a DO probe (along its longitudinal axis) when the DO probe is kept at a fixed angle, is between about 0 degree and about 360 degrees of rotation, between about 0 degree and about 350 degrees of rotation, between about 0 degree and about 340 degrees of rotation, between about 0 degree and about 330 degrees of rotation, between about 0 degree and about 320 degrees of rotation, between about 0 degree and about 310 degrees of rotation, between about 0 degree and about 300 degrees of rotation, between about 0 degree and about 290 degrees of rotation, between about 0 degree and about 280 degrees of rotation, between about 0 degree and about 270 degrees of rotation, between about 0 degree and about 260 degrees of rotation, between about 0 degree and about 250 degrees of rotation, between about 0 degree and about 240 degrees of rotation, between about 0 degree and about 230 degrees of rotation, between about 0 degree and about 220 degrees of rotation, between about 0 degree and about 210 degrees of rotation, between about 0 degree and about 200 degrees of rotation, between about 0 degree and about 190 degrees of rotation, between about 0 degree and about 180 degrees of rotation, between about 0 degree and about 170 degrees of rotation, between about 0 degree and about 160 degrees of rotation, between about 0 degree and about 150 degrees of rotation, between about 0 degree and about 140 degrees of rotation, between about 0 degree and about 130 degrees of rotation, between about 0 degree and about 120 degrees of rotation, between about 0 degree and about 110 degrees of rotation, between about 0 degree and about 100 degrees of rotation, between about 0 degree and about 90 degrees of rotation, between about 0 degree and about 80 degrees of rotation, between about 0 degree and about 70 degrees of rotation, between about 0 degree and about 60 degrees of rotation, between about 0 degree and about 50 degrees of rotation, between about 0 degree and about 40 degrees of rotation, between about 0 degree and about 30 degrees of rotation, between about 10 degrees and about 360 degrees of rotation, between about 10 degrees and about 350 degrees of rotation, between about 10 degrees and about 340 degrees of rotation, between about 10 degrees and about 330 degrees of rotation, between about 10 degrees and about 320 degrees of rotation, between about 10 degrees and about 310 degrees of rotation, between about 10 degrees and about 300 degrees of rotation, between about 10 degrees and about 290 degrees of rotation, between about 10 degrees and about 280 degrees of rotation, between about 10 degrees and about 270 degrees of rotation, between about 10 degrees and about 260 degrees of rotation, between about 10 degrees and about 250 degrees of rotation, between about 10 degrees and about 240 degrees of rotation, between about 10 degrees and about 230 degrees of rotation, between about 10 degrees and about 220 degrees of rotation, between about 10 degrees and about 210 degrees of rotation, between about 10 degrees and about 200 degrees of rotation, between about 10 degrees and about 190 degrees of rotation, between about 10 degrees and about 180 degrees of rotation, between about 10 degrees and about 170 degrees of rotation, between about 10 degrees and about 160 degrees of rotation, between about 10 degrees and about 150 degrees of rotation, between about 10 degrees and about 140 degrees of rotation, between about 10 degrees and about 130 degrees of rotation, between about 10 degrees and about 120 degrees of rotation, between about 10 degrees and about 110 degrees of rotation, between about 10 degrees and about 100 degrees of rotation, between about 10 degrees and about 90 degrees of rotation, between about 10 degrees and about 80 degrees of rotation, between about 10 degrees and about 70 degrees of rotation, between about 10 degrees and about 60 degrees of rotation, between about 10 degrees and about 50 degrees of rotation, between about 10 degrees and about 40 degrees of rotation, between about 10 degrees and about 30 degrees of rotation, between about 20 degrees and about 360 degrees of rotation, between about 20 degrees and about 350 degrees of rotation, between about 20 degrees and about 340 degrees of rotation, between about 20 degrees and about 330 degrees of rotation, between about 20 degrees and about 320 degrees of rotation, between about 20 degrees and about 310 degrees of rotation, between about 20 degrees and about 300 degrees of rotation, between about 20 degrees and about 290 degrees of rotation, between about 20 degrees and about 280 degrees of rotation, between about 20 degrees and about 270 degrees of rotation, between about 20 degrees and about 260 degrees of rotation, between about 20 degrees and about 250 degrees of rotation, between about 20 degrees and about 240 degrees of rotation, between about 20 degree and about 230 degrees of rotation, between about 20 degrees and about 220 degrees of rotation, between about 20 degrees and about 210 degrees of rotation, between about 20 degrees and about 200 degrees of rotation, between about 20 degrees and about 190 degrees of rotation, between about 20 degrees and about 180 degrees of rotation, between about 20 degrees and about 170 degrees of rotation, between about 20 degrees and about 160 degrees of rotation, between about 20 degrees and about 150 degrees of rotation, between about 20 degrees and about 140 degrees of rotation, between about 20 degrees and about 130 degrees of rotation, between about 20 degrees and about 120 degrees of rotation, between about 20 degrees and about 110 degrees of rotation, between about 20 degrees and about 100 degrees of rotation, between about 20 degrees and about 90 degrees of rotation, between about 20 degrees and about 80 degrees of rotation, between about 20 degrees and about 70 degrees of rotation, between about 20 degrees and about 60 degrees of rotation, between about 20 degrees and about 50 degrees of rotation, about 30 degrees and about 360 degrees of rotation, between about 30 degrees and about 350 degrees of rotation, between about 30 degrees and about 340 degrees of rotation, between about 30 degrees and about 330 degrees of rotation, between about 30 degrees and about 320 degrees of rotation, between about 30 degrees and about 310 degrees of rotation, between about 30 degrees and about 300 degrees of rotation, between about 30 degrees and about 290 degrees of rotation, between about 30 degrees and about 280 degrees of rotation, between about 30 degrees and about 270 degrees of rotation, between about 30 degrees and about 260 degrees of rotation, between about 30 degrees and about 250 degrees of rotation, between about 30 degrees and about 240 degrees of rotation, between about 30 degrees and about 230 degrees of rotation, between about 30 degrees and about 220 degrees of rotation, between about 30 degrees and about 210 degrees of rotation, between about 30 degrees and about 200 degrees of rotation, between about 30 degrees and about 190 degrees of rotation, between about 30 degrees and about 180 degrees of rotation, between about 30 degrees and about 170 degrees of rotation, between about 30 degrees and about 160 degrees of rotation, between about 30 degrees and about 150 degrees of rotation, between about 30 degrees and about 140 degrees or rotation, between about 30 degrees and about 130 degrees of rotation, between about 30 degrees and about 120 degrees of rotation, between about 30 degrees and about 110 degrees of rotation, between about 30 degrees and about 100 degrees of rotation, between about 30 degrees and about 90 degrees of rotation, between about 30 degrees and about 80 degrees of rotation, between about 30 degrees and about 70 degrees of rotation, between about 30 degrees and about 60 degrees of rotation, about 40 degrees and about 360 degrees of rotation, between about 40 degrees and about 350 degrees of rotation, between about 40 degrees and about 340 degrees of rotation, between about 40 degrees and about 330 degrees of rotation, between about 40 degrees and about 320 degrees of rotation, between about 40 degrees and about 310 degrees of rotation, between about 40 degrees and about 300 degrees of rotation, between about 40 degrees and about 290 degrees of rotation, between about 40 degrees and about 280 degrees of rotation, between about 40 degrees and about 270 degrees of rotation, between about 40 degrees and about 260 degrees of rotation, between about 40 degrees and about 250 degrees of rotation, between about 40 degrees and about 240 degrees of rotation, between about 40 degrees and about 230 degrees of rotation, between about 40 degrees and about 220 degrees of rotation, between about 40 degrees and about 210 degrees of rotation, between about 40 degrees and about 200 degrees of rotation, between about 40 degrees and about 190 degrees of rotation, between about 40 degrees and about 180 degrees of rotation, between about 40 degrees and about 170 degrees of rotation, between about 40 degrees and about 160 degrees of rotation, between about 40 degrees and about 150 degrees of rotation, between about 40 degrees and about 140 degrees or rotation, between about 40 degrees and about 130 degrees of rotation, between about 40 degrees and about 120 degrees of rotation, between about 40 degrees and about 110 degrees of rotation, between about 40 degrees and about 100 degrees of rotation, between about 40 degrees and about 90 degrees of rotation, between about 40 degrees and about 80 degrees of rotation, between about 40 degrees and about 70 degrees of rotation, about 50 degrees and about 360 degrees of rotation, between about 50 degrees and about 350 degrees of rotation, between about 50 degrees and about 340 degrees of rotation, between about 50 degrees and about 330 degrees of rotation, between about 50 degrees and about 320 degrees of rotation, between about 50 degrees and about 310 degrees of rotation, between about 50 degrees and about 300 degrees of rotation, between about 50 degrees and about 290 degrees of rotation, between about 50 degrees and about 280 degrees of rotation, between about 50 degrees and about 270 degrees of rotation, between about 50 degrees and about 260 degrees of rotation, between about 50 degrees and about 250 degrees of rotation, between about 50 degrees and about 240 degrees of rotation, between about 50 degrees and about 230 degrees of rotation, between about 50 degrees and about 220 degrees of rotation, between about 50 degrees and about 210 degrees of rotation, between about 50 degrees and about 200 degrees of rotation, between about 50 degrees and about 190 degrees of rotation, between about 50 degrees and about 180 degrees of rotation, between about 50 degrees and about 170 degrees of rotation, between about 50 degrees and about 160 degrees of rotation, between about 50 degrees and about 150 degrees of rotation, between about 50 degrees and about 140 degrees or rotation, between about 50 degrees and about 130 degrees of rotation, between about 50 degrees and about 120 degrees of rotation, between about 50 degrees and about 110 degrees of rotation, between about 50 degrees and about 100 degrees of rotation, between about 50 degrees and about 90 degrees of rotation, between about 50 degrees and about 80 degrees of rotation, between about 60 degrees and about 360 degrees of rotation, between about 60 degrees and about 350 degrees of rotation, between about 60 degrees and about 340 degrees of rotation, between about 60 degrees and about 330 degrees of rotation, between about 60 degrees and about 320 degrees of rotation, between about 60 degrees and about 310 degrees of rotation, between about 60 degrees and about 300 degrees of rotation, between about 60 degrees and about 290 degrees of rotation, between about 60 degrees and about 280 degrees of rotation, between about 60 degrees and about 270 degrees of rotation, between about 60 degrees and about 260 degrees of rotation, between about 60 degrees and about 250 degrees of rotation, between about 60 degrees and about 240 degrees of rotation, between about 60 degrees and about 230 degrees of rotation, between about 60 degrees and about 220 degrees of rotation, between about 60 degrees and about 210 degrees of rotation, between about 60 degrees and about 200 degrees of rotation, between about 60 degrees and about 190 degrees of rotation, between about 60 degrees and about 180 degrees of rotation, between about 60 degrees and about 170 degrees of rotation, between about 60 degrees and about 160 degrees of rotation, between about 60 degrees and about 150 degrees of rotation, between about 60 degrees and about 140 degrees of rotation, between about 60 degrees and about 130 degrees of rotation, between about 60 degrees and about 120 degrees of rotation, between about 60 degrees and about 110 degrees of rotation, between about 60 degrees and about 100 degrees of rotation, between about 60 degrees and about 90 degrees of rotation, between about 70 degrees and about 360 degrees of rotation, between about 70 degrees and about 350 degrees of rotation, between about 70 degrees and about 340 degrees of rotation, between about 70 degrees and about 330 degrees of rotation, between about 70 degrees and about 320 degrees of rotation, between about 70 degrees and about 310 degrees of rotation, between about 70 degrees and about 300 degrees of rotation, between about 70 degrees and about 290 degrees of rotation, between about 70 degrees and about 280 degrees of rotation, between about 70 degrees and about 270 degrees of rotation, between about 70 degrees and about 260 degrees of rotation, between about 70 degrees and about 250 degrees of rotation, between about 70 degrees and about 240 degrees of rotation, between about 70 degrees and about 230 degrees of rotation, between about 70 degrees and about 220 degrees of rotation, between about 70 degrees and about 210 degrees of rotation, between about 70 degrees and about 200 degrees of rotation, between about 70 degrees and about 190 degrees of rotation, between about 70 degrees and about 180 degrees of rotation, between about 70 degrees and about 170 degrees of rotation, between about 70 degrees and about 160 degrees of rotation, between about 70 degrees and about 150 degrees of rotation, between about 70 degrees and about 140 degrees of rotation, between about 70 degrees and about 130 degrees of rotation, between about 70 degrees and about 120 degrees of rotation, between about 70 degrees and about 110 degrees of rotation, between about 70 degrees and about 100 degrees of rotation, between about 80 degrees and about 360 degrees of rotation, between about 80 degrees and about 350 degrees of rotation, between about 80 degrees and about 340 degrees of rotation, between about 80 degrees and about 330 degrees of rotation, between about 80 degrees and about 320 degrees of rotation, between about 80 degrees and about 310 degrees of rotation, between about 80 degrees and about 300 degrees of rotation, between about 80 degrees and about 290 degrees of rotation, between about 80 degrees and about 280 degrees of rotation, between about 80 degrees and about 270 degrees of rotation, between about 80 degrees and about 260 degrees of rotation, between about 80 degrees and about 250 degrees of rotation, between about 80 degrees and about 240 degrees of rotation, between about 80 degrees and about 230 degrees of rotation, between about 80 degrees and about 220 degrees of rotation, between about 80 degrees and about 210 degrees of rotation, between about 80 degrees and about 200 degrees of rotation, between about 80 degrees and about 190 degrees of rotation, between about 80 degrees and about 180 degrees of rotation, between about 80 degrees and about 170 degrees of rotation, between about 80 degrees and about 160 degrees of rotation, between about 80 degrees and about 150 degrees of rotation, between about 80 degrees and about 140 degrees of rotation, between about 80 degrees and about 130 degrees of rotation, between about 80 degrees and about 120 degrees of rotation, between about 80 degrees and about 110 degrees of rotation, between about 90 degrees and about 360 degrees of rotation, between about 90 degrees and about 350 degrees of rotation, between about 90 degrees and about 340 degrees of rotation, between about 90 degrees and about 330 degrees of rotation, between about 90 degrees and about 320 degrees of rotation, between about 90 degrees and about 310 degrees of rotation, between about 90 degrees and about 300 degrees of rotation, between about 90 degrees and about 290 degrees of rotation, between about 90 degrees and about 280 degrees of rotation, between about 90 degrees and about 270 degrees of rotation, between about 90 degrees and about 260 degrees of rotation, between about 90 degrees and about 250 degrees of rotation, between about 90 degrees and about 240 degrees of rotation, between about 90 degrees and about 230 degrees of rotation, between about 90 degrees and about 220 degrees of rotation, between about 90 degrees and about 210 degrees of rotation, between about 90 degrees and about 200 degrees of rotation, between about 90 degrees and about 190 degrees of rotation, between about 90 degrees and about 180 degrees of rotation, between about 90 degrees and about 170 degrees of rotation, between about 90 degrees and about 160 degrees of rotation, between about 90 degrees and about 150 degrees of rotation, between about 90 degrees and about 140 degrees of rotation, between about 90 degrees and about 130 degrees of rotation, between about 90 degrees and about 120 degrees of rotation, between about 100 degrees and about 360 degrees of rotation, between about 100 degrees and about 350 degrees of rotation, between about 100 degrees and about 340 degrees of rotation, between about 100 degrees and about 330 degrees of rotation, between about 100 degrees and about 320 degrees of rotation, between about 100 degrees and about 310 degrees of rotation, between about 100 degrees and about 300 degrees of rotation, between about 100 degrees and about 290 degrees of rotation, between about 100 degrees and about 280 degrees of rotation, between about 100 degrees and about 270 degrees of rotation, between about 100 degrees and about 260 degrees of rotation, between about 100 degrees and about 250 degrees of rotation, between about 100 degrees and about 240 degrees of rotation, between about 100 degrees and about 230 degrees of rotation, between about 100 degrees and about 220 degrees of rotation, between about 100 degrees and about 210 degrees of rotation, between about 100 degrees and about 200 degrees of rotation, between about 100 degrees and about 190 degrees of rotation, between about 100 degrees and about 180 degrees of rotation, between about 100 degrees and about 170 degrees of rotation, between about 100 degrees and about 160 degrees of rotation, between about 100 degrees and about 150 degrees of rotation, between about 100 degrees and about 140 degrees of rotation, between about 100 degrees and about 130 degrees of rotation, between about 110 degrees and about 360 degrees of rotation, between about 110 degrees and about 350 degrees of rotation, between about 110 degrees and about 340 degrees of rotation, between about 110 degrees and about 330 degrees of rotation, between about 110 degrees and about 320 degrees of rotation, between about 110 degrees and about 310 degrees of rotation, between about 110 degrees and about 300 degrees of rotation, between about 110 degrees and about 290 degrees of rotation, between about 110 degrees and about 280 degrees of rotation, between about 110 degrees and about 270 degrees of rotation, between about 110 degrees and about 260 degrees of rotation, between about 110 degrees and about 250 degrees of rotation, between about 110 degrees and about 240 degrees of rotation, between about 110 degrees and about 230 degrees of rotation, between about 110 degrees and about 220 degrees of rotation, between about 110 degrees and about 210 degrees of rotation, between about 110 degrees and about 200 degrees of rotation, between about 110 degrees and about 190 degrees of rotation, between about 110 degrees and about 180 degrees of rotation, between about 110 degrees and about 170 degrees of rotation, between about 110 degrees and about 160 degrees of rotation, between about 110 degrees and about 150 degrees of rotation, between about 110 degrees and about 140 degrees of rotation, between about 120 degrees and about 360 degrees of rotation, between about 120 degrees and about 350 degrees of rotation, between about 120 degrees and about 340 degrees of rotation, between about 120 degrees and about 330 degrees of rotation, between about 120 degrees and about 320 degrees of rotation, between about 120 degrees and about 310 degrees of rotation, between about 120 degrees and about 300 degrees of rotation, between about 120 degrees and about 290 degrees of rotation, between about 120 degrees and about 280 degrees of rotation, between about 120 degrees and about 270 degrees of rotation, between about 120 degrees and about 260 degrees of rotation, between about 120 degrees and about 250 degrees of rotation, between about 120 degrees and about 240 degrees of rotation, between about 120 degrees and about 230 degrees of rotation, between about 120 degrees and about 220 degrees of rotation, between about 120 degrees and about 210 degrees of rotation, between about 120 degrees and about 200 degrees of rotation, between about 120 degrees and about 190 degrees of rotation, between about 120 degrees and about 180 degrees of rotation, between about 120 degrees and about 170 degrees of rotation, between about 120 degrees and about 160 degrees of rotation, between about 120 degrees and about 150 degrees of rotation, between about 130 degrees and about 360 degrees of rotation, between about 130 degrees and about 350 degrees of rotation, between about 130 degrees and about 340 degrees of rotation, between about 130 degrees and about 330 degrees of rotation, between about 130 degrees and about 320 degrees of rotation, between about 130 degrees and about 310 degrees of rotation, between about 130 degrees and about 300 degrees of rotation, between about 130 degrees and about 290 degrees of rotation, between about 130 degrees and about 280 degrees of rotation, between about 130 degrees and about 270 degrees of rotation, between about 130 degrees and about 260 degrees of rotation, between about 130 degrees and about 250 degrees of rotation, between about 130 degrees and about 240 degrees of rotation, between about 130 degrees and about 230 degrees of rotation, between about 130 degrees and about 220 degrees of rotation, between about 130 degrees and about 210 degrees of rotation, between about 130 degrees and about 200 degrees of rotation, between about 130 degrees and about 190 degrees of rotation, between about 130 degrees and about 180 degrees of rotation, between about 130 degrees and about 170 degrees of rotation, between about 130 degrees and about 160 degrees of rotation, between about 140 degrees and about 360 degrees of rotation, between about 140 degrees and about 350 degrees of rotation, between about 140 degrees and about 340 degrees of rotation, between about 140 degrees and about 330 degrees of rotation, between about 140 degrees and about 320 degrees of rotation, between about 140 degrees and about 310 degrees of rotation, between about 140 degrees and about 300 degrees of rotation, between about 140 degrees and about 290 degrees of rotation, between about 140 degrees and about 280 degrees of rotation, between about 140 degrees and about 270 degrees of rotation, between about 140 degrees and about 260 degrees of rotation, between about 140 degrees and about 250 degrees of rotation, between about 140 degrees and about 240 degrees of rotation, between about 140 degrees and about 230 degrees of rotation, between about 140 degrees and about 220 degrees of rotation, between about 140 degrees and about 210 degrees of rotation, between about 140 degrees and about 200 degrees of rotation, between about 140 degrees and about 190 degrees of rotation, between about 140 degrees and about 180 degrees of rotation, between about 140 degrees and about 170 degrees of rotation, between about 150 degrees and about 360 degrees of rotation, between about 150 degrees and about 350 degrees of rotation, between about 150 degrees and about 340 degrees of rotation, between about 150 degrees and about 330 degrees of rotation, between about 150 degrees and about 320 degrees of rotation, between about 150 degrees and about 310 degrees of rotation, between about 150 degrees and about 300 degrees of rotation, between about 150 degrees and about 290 degrees of rotation, between about 150 degrees and about 280 degrees of rotation, between about 150 degrees and about 270 degrees of rotation, between about 150 degrees and about 260 degrees of rotation, between about 150 degrees and about 250 degrees of rotation, between about 150 degrees and about 240 degrees of rotation, between about 150 degrees and about 230 degrees of rotation, between about 150 degrees and about 220 degrees of rotation, between about 150 degrees and about 210 degrees of rotation, between about 150 degrees and about 200 degrees of rotation, between about 150 degrees and about 190 degrees of rotation, between about 150 degrees and about 180 degrees of rotation, between about 160 degrees and about 360 degrees of rotation, between about 160 degrees and about 350 degrees of rotation, between about 160 degrees and about 340 degrees of rotation, between about 160 degrees and about 330 degrees of rotation, between about 160 degrees and about 320 degrees of rotation, between about 160 degrees and about 310 degrees of rotation, between about 160 degrees and about 300 degrees of rotation, between about 160 degrees and about 290 degrees of rotation, between about 160 degrees and about 280 degrees of rotation, between about 160 degrees and about 270 degrees of rotation, between about 160 degrees and about 260 degrees of rotation, between about 160 degrees and about 250 degrees of rotation, between about 160 degrees and about 240 degrees of rotation, between about 160 degrees and about 230 degrees of rotation, between about 160 degrees and about 220 degrees of rotation, between about 160 degrees and about 210 degrees of rotation, between about 160 degrees and about 200 degrees of rotation, between about 160 degrees and about 190 degrees of rotation, between about 170 degrees and about 360 degrees of rotation, between about 170 degrees and about 350 degrees of rotation, between about 170 degrees and about 340 degrees of rotation, between about 170 degrees and about 330 degrees of rotation, between about 170 degrees and about 320 degrees of rotation, between about 170 degrees and about 310 degrees of rotation, between about 170 degrees and about 300 degrees of rotation, between about 170 degrees and about 290 degrees of rotation, between about 170 degrees and about 280 degrees of rotation, between about 170 degrees and about 270 degrees of rotation, between about 170 degrees and about 260 degrees of rotation, between about 170 degrees and about 250 degrees of rotation, between about 170 degrees and about 240 degrees of rotation, between about 170 degrees and about 230 degrees of rotation, between about 170 degrees and about 220 degrees of rotation, between about 170 degrees and about 210 degrees of rotation, between about 170 degrees and about 200 degrees of rotation, between about 180 degrees and about 360 degrees of rotation, between about 180 degrees and about 350 degrees of rotation, between about 180 degrees and about 340 degrees of rotation, between about 180 degrees and about 330 degrees of rotation, between about 180 degrees and about 320 degrees of rotation, between about 180 degrees and about 310 degrees of rotation, between about 180 degrees and about 300 degrees of rotation, between about 180 degrees and about 290 degrees of rotation, between about 180 degrees and about 280 degrees of rotation, between about 180 degrees and about 270 degrees of rotation, between about 180 degrees and about 260 degrees of rotation, between about 180 degrees and about 250 degrees of rotation, between about 180 degrees and about 240 degrees of rotation, between about 180 degrees and about 230 degrees of rotation, between about 180 degrees and about 220 degrees of rotation, between about 180 degrees and about 210 degrees of rotation, between about 190 degrees and about 360 degrees of rotation, between about 190 degrees and about 350 degrees of rotation, between about 190 degrees and about 340 degrees of rotation, between about 190 degrees and about 330 degrees of rotation, between about 190 degrees and about 320 degrees of rotation, between about 190 degrees and about 310 degrees of rotation, between about 190 degrees and about 300 degrees of rotation, between about 190 degrees and about 290 degrees of rotation, between about 190 degrees and about 280 degrees of rotation, between about 190 degrees and about 270 degrees of rotation, between about 190 degrees and about 260 degrees of rotation, between about 190 degrees and about 250 degrees of rotation, between about 190 degrees and about 240 degrees of rotation, between about 190 degrees and about 230 degrees of rotation, between about 190 degrees and about 220 degrees of rotation, between about 200 degrees and about 360 degrees of rotation, between about 200 degrees and about 350 degrees of rotation, between about 200 degrees and about 340 degrees of rotation, between about 200 degrees and about 330 degrees of rotation, between about 200 degrees and about 320 degrees of rotation, between about 200 degrees and about 310 degrees of rotation, between about 200 degrees and about 300 degrees of rotation, between about 200 degrees and about 290 degrees of rotation, between about 200 degrees and about 280 degrees of rotation, between about 200 degrees and about 270 degrees of rotation, between about 200 degrees and about 260 degrees of rotation, between about 200 degrees and about 250 degrees of rotation, between about 200 degrees and about 240 degrees of rotation, between about 200 degrees and about 230 degrees of rotation, between about 210 degrees and about 360 degrees of rotation, between about 210 degrees and about 350 degrees of rotation, between about 210 degrees and about 340 degrees of rotation, between about 210 degrees and about 330 degrees of rotation, between about 210 degrees and about 320 degrees of rotation, between about 210 degrees and about 310 degrees of rotation, between about 210 degrees and about 300 degrees of rotation, between about 210 degrees and about 290 degrees of rotation, between about 210 degrees and about 280 degrees of rotation, between about 210 degrees and about 270 degrees of rotation, between about 210 degrees and about 260 degrees of rotation, between about 210 degrees and about 250 degrees of rotation, between about 210 degrees and about 240 degrees of rotation, between about 220 degrees and about 360 degrees of rotation, between about 220 degrees and about 350 degrees of rotation, between about 220 degrees and about 340 degrees of rotation, between about 220 degrees and about 330 degrees of rotation, between about 220 degrees and about 320 degrees of rotation, between about 220 degrees and about 310 degrees of rotation, between about 220 degrees and about 300 degrees of rotation, between about 220 degrees and about 290 degrees of rotation, between about 220 degrees and about 280 degrees of rotation, between about 220 degrees and about 270 degrees of rotation, between about 220 degrees and about 260 degrees of rotation, between about 220 degrees and about 250 degrees of rotation, between about 230 degrees and about 360 degrees of rotation, between about 230 degrees and about 350 degrees of rotation, between about 230 degrees and about 340 degrees of rotation, between about 230 degrees and about 330 degrees of rotation, between about 230 degrees and about 320 degrees of rotation, between about 230 degrees and about 310 degrees of rotation, between about 230 degrees and about 300 degrees of rotation, between about 230 degrees and about 290 degrees of rotation, between about 230 degrees and about 280 degrees of rotation, between about 230 degrees and about 270 degrees of rotation, between about 230 degrees and about 260 degrees of rotation, between about 240 degrees and about 360 degrees of rotation, between about 240 degrees and about 350 degrees of rotation, between about 240 degrees and about 340 degrees of rotation, between about 240 degrees and about 330 degrees of rotation, between about 240 degrees and about 320 degrees of rotation, between about 240 degrees and about 310 degrees of rotation, between about 240 degrees and about 300 degrees of rotation, between about 240 degrees and about 290 degrees of rotation, between about 240 degrees and about 280 degrees of rotation, between about 240 degrees and about 270 degrees of rotation, between about 250 degrees and about 360 degrees of rotation, between about 250 degrees and about 350 degrees of rotation, between about 250 degrees and about 340 degrees of rotation, between about 250 degrees and about 330 degrees of rotation, between about 250 degrees and about 320 degrees of rotation, between about 250 degrees and about 310 degrees of rotation, between about 250 degrees and about 300 degrees of rotation, between about 250 degrees and about 290 degrees of rotation, between about 250 degrees and about 280 degrees of rotation, between about 260 degrees and about 360 degrees of rotation, between about 260 degrees and about 350 degrees of rotation, between about 260 degrees and about 340 degrees of rotation, between about 260 degrees and about 330 degrees of rotation, between about 260 degrees and about 320 degrees of rotation, between about 260 degrees and about 310 degrees of rotation, between about 260 degrees and about 300 degrees of rotation, between about 260 degrees and about 290 degrees of rotation, between about 270 degrees and about 360 degrees of rotation, between about 270 degrees and about 350 degrees of rotation, between about 270 degrees and about 340 degrees of rotation, between about 270 degrees and about 330 degrees of rotation, between about 270 degrees and about 320 degrees of rotation, between about 270 degrees and about 310 degrees of rotation, between about 270 degrees and about 300 degrees of rotation, between about 280 degrees and about 360 degrees of rotation, between about 280 degrees and about 350 degrees of rotation, between about 280 degrees and about 340 degrees of rotation, between about 280 degrees and about 330 degrees of rotation, between about 280 degrees and about 320 degrees of rotation, between about 280 degrees and about 310 degrees of rotation, between about 290 degrees and about 360 degrees of rotation, between about 290 degrees and about 350 degrees of rotation, between about 290 degrees and about 340 degrees of rotation, between about 290 degrees and about 330 degrees of rotation, between about 290 degrees and about 320 degrees of rotation, between about 300 degrees and about 360 degrees of rotation, between about 300 degrees and about 350 degrees of rotation, between about 300 degrees and about 340 degrees of rotation, between about 300 degrees and about 330 degrees of rotation, or between about 330 degrees and about 360 degrees of rotation.

In some embodiments, the plurality of different fixed orientations is a plurality of different rotations of the DO probe, e.g., between about 0 degree and about 360 degrees of rotation, between about 0 degree and about 350 degrees of rotation, between about 0 degree and about 340 degrees of rotation, between about 0 degree and about 330 degrees of rotation, between about 0 degree and about 320 degrees of rotation, between about 0 degree and about 310 degrees of rotation, between about 0 degree and about 300 degrees of rotation, between about 0 degree and about 290 degrees of rotation, between about 0 degree and about 280 degrees of rotation, between about 0 degree and about 270 degrees of rotation, between about 0 degree and about 260 degrees of rotation, between about 0 degree and about 250 degrees of rotation, between about 0 degree and about 240 degrees of rotation, between about 0 degree and about 230 degrees of rotation, between about 0 degree and about 220 degrees of rotation, between about 0 degree and about 210 degrees of rotation, between about 0 degree and about 200 degrees of rotation, between about 0 degree and about 190 degrees of rotation, between about 0 degree and about 180 degrees of rotation, between about 0 degree and about 170 degrees of rotation, between about 0 degree and about 160 degrees of rotation, between about 0 degree and about 150 degrees of rotation, between about 0 degree and about 140 degrees of rotation, between about 0 degree and about 130 degrees of rotation, between about 0 degree and about 120 degrees of rotation, between about 0 degree and about 110 degrees of rotation, between about 0 degree and about 100 degrees of rotation, between about 0 degree and about 90 degrees of rotation, between about 0 degree and about 80 degrees of rotation, between about 0 degree and about 70 degrees of rotation, between about 0 degree and about 60 degrees of rotation, between about 0 degree and about 50 degrees of rotation, between about 0 degree and about 40 degrees of rotation, between about 0 degree and about 30 degrees of rotation, between about 0 degree and about 20 degrees of rotation, between about 0 degree and about 10 degrees of rotation, about 30 degrees and about 360 degrees of rotation, between about 30 degrees and about 350 degrees of rotation, between about 30 degrees and about 340 degrees of rotation, between about 30 degrees and about 330 degrees of rotation, between about 30 degrees and about 320 degrees of rotation, between about 30 degrees and about 310 degrees of rotation, between about 30 degrees and about 300 degrees of rotation, between about 30 degrees and about 290 degrees of rotation, between about 30 degrees and about 280 degrees of rotation, between about 30 degrees and about 270 degrees of rotation, between about 30 degrees and about 260 degrees of rotation, between about 30 degrees and about 250 degrees of rotation, between about 30 degrees and about 240 degrees of rotation, between about 30 degrees and about 230 degrees of rotation, between about 30 degrees and about 220 degrees of rotation, between about 30 degrees and about 210 degrees of rotation, between about 30 degrees and about 200 degrees of rotation, between about 30 degrees and about 190 degrees of rotation, between about 30 degrees and about 180 degrees of rotation, between about 30 degrees and about 170 degrees of rotation, between about 30 degrees and about 160 degrees of rotation, between about 30 degrees and about 150 degrees of rotation, between about 30 degrees and about 140 degrees of rotation, between about 30 degrees and about 130 degrees of rotation, between about 30 degrees and about 120 degrees of rotation, between about 30 degrees and about 110 degrees of rotation, between about 30 degrees and about 100 degrees of rotation, between about 30 degrees and about 90 degrees of rotation, between about 30 degrees and about 80 degrees of rotation, between about 30 degrees and about 70 degrees of rotation, between about 30 degrees and about 60 degrees of rotation, between about 30 degrees and about 50 degrees of rotation, between about 30 degrees and about 40 degrees of rotation, between about 60 degrees and about 360 degrees of rotation, between about 60 degrees and about 350 degrees of rotation, between about 60 degrees and about 340 degrees of rotation, between about 60 degrees and about 330 degrees of rotation, between about 60 degrees and about 320 degrees of rotation, between about 60 degrees and about 310 degrees of rotation, between about 60 degrees and about 300 degrees of rotation, between about 60 degrees and about 290 degrees of rotation, between about 60 degrees and about 280 degrees of rotation, between about 60 degrees and about 270 degrees of rotation, between about 60 degrees and about 260 degrees of rotation, between about 60 degrees and about 250 degrees of rotation, between about 60 degrees and about 240 degrees of rotation, between about 60 degrees and about 230 degrees of rotation, between about 60 degrees and about 220 degrees of rotation, between about 60 degrees and about 210 degrees of rotation, between about 60 degrees and about 200 degrees of rotation, between about 60 degrees and about 190 degrees of rotation, between about 60 degrees and about 180 degrees of rotation, between about 60 degrees and about 170 degrees of rotation, between about 60 degrees and about 160 degrees of rotation, between about 60 degrees and about 150 degrees of rotation, between about 60 degrees and about 140 degrees of rotation, between about 60 degrees and about 130 degrees of rotation, between about 60 degrees and about 120 degrees of rotation, between about 60 degrees and about 110 degrees of rotation, between about 60 degrees and about 100 degrees of rotation, between about 60 degrees and about 90 degrees of rotation, between about 60 degrees and about 80 degrees of rotation, between about 60 degrees and about 70 degrees of rotation, between about 90 degrees and about 360 degrees of rotation, between about 90 degrees and about 350 degrees of rotation, between about 90 degrees and about 340 degrees of rotation, between about 90 degrees and about 330 degrees of rotation, between about 90 degrees and about 320 degrees of rotation, between about 90 degrees and about 310 degrees of rotation, between about 90 degrees and about 300 degrees of rotation, between about 90 degrees and about 290 degrees of rotation, between about 90 degrees and about 280 degrees of rotation, between about 90 degrees and about 270 degrees of rotation, between about 90 degrees and about 260 degrees of rotation, between about 90 degrees and about 250 degrees of rotation, between about 90 degrees and about 240 degrees of rotation, between about 90 degrees and about 230 degrees of rotation, between about 90 degrees and about 220 degrees of rotation, between about 90 degrees and about 210 degrees of rotation, between about 90 degrees and about 200 degrees of rotation, between about 90 degrees and about 190 degrees of rotation, between about 90 degrees and about 180 degrees of rotation, between about 90 degrees and about 170 degrees of rotation, between about 90 degrees and about 160 degrees of rotation, between about 90 degrees and about 150 degrees of rotation, between about 90 degrees and about 140 degrees of rotation, between about 90 degrees and about 130 degrees of rotation, between about 90 degrees and about 120 degrees of rotation, between about 90 degrees and about 110 degrees of rotation, between about 90 degrees and about 100 degrees of rotation, between about 120 degrees and about 360 degrees of rotation, between about 120 degrees and about 350 degrees of rotation, between about 120 degrees and about 340 degrees of rotation, between about 120 degrees and about 330 degrees of rotation, between about 120 degrees and about 320 degrees of rotation, between about 120 degrees and about 310 degrees of rotation, between about 120 degrees and about 300 degrees of rotation, between about 120 degrees and about 290 degrees of rotation, between about 120 degrees and about 280 degrees of rotation, between about 120 degrees and about 270 degrees of rotation, between about 120 degrees and about 260 degrees of rotation, between about 120 degrees and about 250 degrees of rotation, between about 120 degrees and about 240 degrees of rotation, between about 120 degrees and about 230 degrees of rotation, between about 120 degrees and about 220 degrees of rotation, between about 120 degrees and about 210 degrees of rotation, between about 120 degrees and about 200 degrees of rotation, between about 120 degrees and about 190 degrees of rotation, between about 120 degrees and about 180 degrees of rotation, between about 120 degrees and about 170 degrees of rotation, between about 120 degrees and about 160 degrees of rotation, between about 120 degrees and about 150 degrees of rotation, between about 120 degrees and about 140 degrees of rotation, between about 120 degrees and about 130 degrees of rotation, between about 150 degrees and about 360 degrees of rotation, between about 150 degrees and about 350 degrees of rotation, between about 150 degrees and about 340 degrees of rotation, between about 150 degrees and about 330 degrees of rotation, between about 150 degrees and about 320 degrees of rotation, between about 150 degrees and about 310 degrees of rotation, between about 150 degrees and about 300 degrees of rotation, between about 150 degrees and about 290 degrees of rotation, between about 150 degrees and about 280 degrees of rotation, between about 150 degrees and about 270 degrees of rotation, between about 150 degrees and about 260 degrees of rotation, between about 150 degrees and about 250 degrees of rotation, between about 150 degrees and about 240 degrees of rotation, between about 150 degrees and about 230 degrees of rotation, between about 150 degrees and about 220 degrees of rotation, between about 150 degrees and about 210 degrees of rotation, between about 150 degrees and about 200 degrees of rotation, between about 150 degrees and about 190 degrees of rotation, between about 150 degrees and about 180 degrees of rotation, between about 150 degrees and about 170 degrees of rotation, between about 150 degrees and about 160 degrees of rotation, between about 180 degrees and about 360 degrees of rotation, between about 180 degrees and about 350 degrees of rotation, between about 180 degrees and about 340 degrees of rotation, between about 180 degrees and about 330 degrees of rotation, between about 180 degrees and about 320 degrees of rotation, between about 180 degrees and about 310 degrees of rotation, between about 180 degrees and about 300 degrees of rotation, between about 180 degrees and about 290 degrees of rotation, between about 180 degrees and about 280 degrees of rotation, between about 180 degrees and about 270 degrees of rotation, between about 180 degrees and about 260 degrees of rotation, between about 180 degrees and about 250 degrees of rotation, between about 180 degrees and about 240 degrees of rotation, between about 180 degrees and about 230 degrees of rotation, between about 180 degrees and about 220 degrees of rotation, between about 180 degrees and about 210 degrees of rotation, between about 180 degrees and about 200 degrees of rotation, between about 180 degrees and about 190 degrees of rotation, between about 210 degrees and about 360 degrees of rotation, between about 210 degrees and about 350 degrees of rotation, between about 210 degrees and about 340 degrees of rotation, between about 210 degrees and about 330 degrees of rotation, between about 210 degrees and about 320 degrees of rotation, between about 210 degrees and about 310 degrees of rotation, between about 210 degrees and about 300 degrees of rotation, between about 210 degrees and about 290 degrees of rotation, between about 210 degrees and about 280 degrees of rotation, between about 210 degrees and about 270 degrees of rotation, between about 210 degrees and about 260 degrees of rotation, between about 210 degrees and about 250 degrees of rotation, between about 210 degrees and about 240 degrees of rotation, between about 210 degrees and about 230 degrees of rotation, between about 210 degrees and about 220 degrees of rotation, between about 240 degrees and about 360 degrees of rotation, between about 240 degrees and about 350 degrees of rotation, between about 240 degrees and about 340 degrees of rotation, between about 240 degrees and about 330 degrees of rotation, between about 240 degrees and about 320 degrees of rotation, between about 240 degrees and about 310 degrees of rotation, between about 240 degrees and about 300 degrees of rotation, between about 240 degrees and about 290 degrees of rotation, between about 240 degrees and about 280 degrees of rotation, between about 240 degrees and about 270 degrees of rotation, between about 240 degrees and about 260 degrees of rotation, between about 240 degrees and about 250 degrees of rotation, between about 270 degrees and about 360 degrees of rotation, between about 270 degrees and about 350 degrees of rotation, between about 270 degrees and about 340 degrees of rotation, between about 270 degrees and about 330 degrees of rotation, between about 270 degrees and about 320 degrees of rotation, between about 270 degrees and about 310 degrees of rotation, between about 270 degrees and about 300 degrees of rotation, between about 270 degrees and about 290 degrees of rotation, between about 270 degrees and about 280 degrees of rotation, between about 300 degrees and about 360 degrees of rotation, between about 300 degrees and about 350 degrees of rotation, between about 300 degrees and about 340 degrees of rotation, between about 300 degrees and about 330 degrees of rotation, between about 300 degrees and about 320 degrees of rotation, between about 300 degrees and about 310 degrees of rotation, between about 330 degrees and about 360 degrees of rotation, between about 330 degrees and about 350 degrees of rotation, between about 330 degrees and about 340 degrees of rotation, between about 340 degrees and about 360 degrees of rotation, between about 340 degrees and about 350 degrees of rotation, or between about 350 degrees and about 360 degrees of rotation, where the DO probe is positioned in a plurality of different fixed angles (e.g., any of the exemplary fixed angles described herein or any of the exemplary ranges of fixed angles described herein). Any one or more fixed angles can be used to identify the plurality of fixed orientations.

The degree of rotation described herein is along the longitudinal axis of the DO probe as illustrated in FIGS. 4A-4B and the direction of the rotation is as shown in FIGS. 4A-4B. A DO probe having a fixed angle of 90 degrees and having a 0-degree rotation is illustrated in FIG. 4A. A DO probe having a fixed angle of 90 degrees and having a 180-degree rotation is illustrated in FIG. 4B.

In some embodiments, the orientation of the DO probe is defined in a three-dimensional context. For example, an “upward” position of the DO probe refers to an orientation where the DO probe is placed in a fixed angle of about 60-90 degrees (e.g., about 60-70 degrees) with a 0-degree rotation (as illustrated in FIG. 4A). In some embodiments, a “right” position of the DO probe refers to an orientation where the DO probe is placed in a fixed angle of about 60-90 degrees (e.g., about 60-70 degrees) with a 90-degree rotation in the direction illustrated in FIGS. 4A-4B. A “downward” position of the DO probe refers to an orientation where the DO probe is placed in a fixed angle of about 60-90 degrees (e.g., about 60-70 degrees) with a 180-degree rotation as illustrated in FIG. 4B. In some embodiments, a “left” position of the DO probe refers to an orientation where the DO probe is placed in a fixed angle of about 60-90 degrees (e.g., about 60-70) with a 270-degree rotation in the direction illustrated in FIGS. 4A-4B. Any other commonly referred positions should be readily understood by a person of ordinary skill in the art in view of the illustration in FIGS. 3A-3E and FIGS. 4A-4B.

The plurality of different fixed orientations can be about 1, 2, 3, 4, 5, 6, 7, 8, 9, or 10 different fixed orientations. The selection of the fixed orientation for the subsequent mammalian cell culture can be based on any suitable criteria. In some embodiments, the selection of the fixed orientation is based on the DO measurement from the DO probes in different fixed orientations. For example, a fixed orientation may be selected because it is minimally impacted by the accumulation of air bubbles on or near the sensor. For example, a fixed orientation can be selected because it does not result in falsely increased and/or falsely decreased measurements of the DO level (as determined by analyzing the measurements of DO level in the bioreactor by the DO probe positioned at the plurality of different orientations).

The accumulation of air bubbles on or near the sensor of the DO probes can be a cause of inaccurate DO measurement by the DO probes. When air bubbles accumulate on or near the sensor, they may cause falsely increased DO measurement by the DO probe. In some embodiments, the selected fixed orientation of the DO probe reduces accumulation of air bubbles on or near the sensor of the DO probe. In some embodiments, the measurement by the DO probe in the selected fixed orientation is essentially free from the impact of accumulated air bubbles. In some embodiments, the DO measurement by the DO probe in the selected fixed orientation is lower compared to the DO measurement by a DO probe in another orientation, when the measurements are conducted in the same bioreactor under otherwise same conditions.

The method described herein also includes adjusting one or more culturing conditions based on the DO measurement of the DO probe in the selected fixed orientation to achieve the selected DO level. In some embodiments, the modifying of the one or more culturing conditions includes adjusting one or more threshold parameters in the cell culture medium. These threshold parameters include, without limitation, optical density (OD), dissolved oxygen (DO), pH, concentration of nutrient in the culture medium, total concentration of the first carbon source added to the culture medium, or any combination thereof. In some embodiments, the modifying of the one or more culturing conditions includes filtering out the reducing agents (byproducts of reduction reactions) from the cell culture medium.

In some embodiments, the volumetric input power is adjusted or optimized to achieve the selected DO level. In some embodiments, the culturing temperature is adjusted or optimized to achieve the selected DO level. In some embodiments, the culture volume is adjusted to achieve the selected DO level.

The method described herein can also be used in a pilot scale process and/or a scale-up process for mammalian cell culture and protein (e.g. antibody) production. Depending on the process, the internal volume of the perfusion bioreactor can be different. In some embodiments, the internal volume of the perfusion bioreactor is about 1 L to about 1000 L. In some embodiments, the internal volume of the perfusion bioreactor is about 1 L to about 1000 L. In some embodiments, the internal volume of the perfusion bioreactor is about 1 L to about 900 L. In some embodiments, the internal volume of the perfusion bioreactor is about 1 L to about 800 L. In some embodiments, the internal volume of the perfusion bioreactor is about 1 L to about 700 L. In some embodiments, the internal volume of the perfusion bioreactor is about 1 L to about 600 L. In some embodiments, the internal volume of the perfusion bioreactor is about 1 L to about 500 L. In some embodiments, the internal volume of the perfusion bioreactor is about 1 L to about 400 L. In some embodiments, the internal volume of the perfusion bioreactor is about 1 L to about 300 L. In some embodiments, the internal volume of the perfusion bioreactor is about 1 L to about 200 L. In some embodiments, the internal volume of the perfusion bioreactor is about 1 L to about 100 L. In some embodiments, the internal volume of the perfusion bioreactor is about 1 L to about 90 L. In some embodiments, the internal volume of the perfusion bioreactor is about 1 L to about 80 L. In some embodiments, the internal volume of the perfusion bioreactor is about 1 L to about 70 L. In some embodiments, the internal volume of the perfusion bioreactor is about 1 L to about 60 L. In some embodiments, the internal volume of the perfusion bioreactor is about 1 L to about 50 L. In some embodiments, the internal volume of the perfusion bioreactor is about 1 L to about 40 L. In some embodiments, the internal volume of the perfusion bioreactor is about 1 L to about 30 L. In some embodiments, the internal volume of the perfusion bioreactor is about 1 L to about 20 L. In some embodiments, the internal volume of the perfusion bioreactor is about 1 L to about 10 L. In some embodiments, the internal volume of the perfusion bioreactor is about 1 L to about 9 L. In some embodiments, the internal volume of the perfusion bioreactor is about 1 L to about 8 L. In some embodiments, the internal volume of the perfusion bioreactor is about 1 L to about 7 L. In some embodiments, the internal volume of the perfusion bioreactor is about 1 L to about 6 L. In some embodiments, the internal volume of the perfusion bioreactor is about 1 L to about 5 L.

In some embodiments, the internal volume of the perfusion bioreactor is about 5 L to about 1000 L. In some embodiments, the internal volume of the perfusion bioreactor is about 5 L to about 900 L. In some embodiments, the internal volume of the perfusion bioreactor is about 5 L to about 800 L. In some embodiments, the internal volume of the perfusion bioreactor is about 5 L to about 700 L. In some embodiments, the internal volume of the perfusion bioreactor is about 5 L to about 600 L. In some embodiments, the internal volume of the perfusion bioreactor is about 5 L to about 500 L. In some embodiments, the internal volume of the perfusion bioreactor is about 5 L to about 400 L. In some embodiments, the internal volume of the perfusion bioreactor is about 5 L to about 300 L. In some embodiments, the internal volume of the perfusion bioreactor is about 5 L to about 200 L. In some embodiments, the internal volume of the perfusion bioreactor is about 5 L to about 100 L. In some embodiments, the internal volume of the perfusion bioreactor is about 5 L to about 90 L. In some embodiments, the internal volume of the perfusion bioreactor is about 5 L to about 80 L. In some embodiments, the internal volume of the perfusion bioreactor is about 5 L to about 70 L. In some embodiments, the internal volume of the perfusion bioreactor is about 5 L to about 60 L. In some embodiments, the internal volume of the perfusion bioreactor is about 5 L to about 50 L. In some embodiments, the internal volume of the perfusion bioreactor is about 5 L to about 40 L. In some embodiments, the internal volume of the perfusion bioreactor is about 5 L to about 30 L. In some embodiments, the internal volume of the perfusion bioreactor is about 5 L to about 20 L. In some embodiments, the internal volume of the perfusion bioreactor is about 5 L to about 10 L.

In some embodiments, the internal volume of the perfusion bioreactor is about 10 L to about 1000 L. In some embodiments, the internal volume of the perfusion bioreactor is about 10 L to about 900 L. In some embodiments, the internal volume of the perfusion bioreactor is about 10 L to about 800 L. In some embodiments, the internal volume of the perfusion bioreactor is about 10 L to about 700 L. In some embodiments, the internal volume of the perfusion bioreactor is about 10 L to about 600 L. In some embodiments, the internal volume of the perfusion bioreactor is about 10 L to about 500 L. In some embodiments, the internal volume of the perfusion bioreactor is about 10 L to about 400 L. In some embodiments, the internal volume of the perfusion bioreactor is about 10 L to about 300 L. In some embodiments, the internal volume of the perfusion bioreactor is about 10 L to about 200 L. In some embodiments, the internal volume of the perfusion bioreactor is about 10 L to about 100 L. In some embodiments, the internal volume of the perfusion bioreactor is about 10 L to about 90 L. In some embodiments, the internal volume of the perfusion bioreactor is about 10 L to about 80 L. In some embodiments, the internal volume of the perfusion bioreactor is about 10 L to about 70 L. In some embodiments, the internal volume of the perfusion bioreactor is about 10 L to about 60 L. In some embodiments, the internal volume of the perfusion bioreactor is about 10 L to about 50 L. In some embodiments, the internal volume of the perfusion bioreactor is about 10 L to about 40 L. In some embodiments, the internal volume of the perfusion bioreactor is about 10 L to about 30 L. In some embodiments, the internal volume of the perfusion bioreactor is about 10 L to about 20 L.

In some embodiments, the internal volume of the perfusion bioreactor is about 50 L to about 1000 L. In some embodiments, the internal volume of the perfusion bioreactor is about 50 L to about 900 L. In some embodiments, the internal volume of the perfusion bioreactor is about 50 L to about 800 L. In some embodiments, the internal volume of the perfusion bioreactor is about 50 L to about 700 L. In some embodiments, the internal volume of the perfusion bioreactor is about 50 L to about 600 L. In some embodiments, the internal volume of the perfusion bioreactor is about 50 L to about 500 L. In some embodiments, the internal volume of the perfusion bioreactor is about 50 L to about 400 L. In some embodiments, the internal volume of the perfusion bioreactor is about 50 L to about 300 L. In some embodiments, the internal volume of the perfusion bioreactor is about 50 L to about 200 L. In some embodiments, the internal volume of the perfusion bioreactor is about 50 L to about 100 L.

In some embodiments, the internal volume of the perfusion bioreactor is about 100 L to about 1000 L. In some embodiments, the internal volume of the perfusion bioreactor is about 100 L to about 900 L. In some embodiments, the internal volume of the perfusion bioreactor is about 100 L to about 800 L. In some embodiments, the internal volume of the perfusion bioreactor is about 100 L to about 700 L. In some embodiments, the internal volume of the perfusion bioreactor is about 100 L to about 600 L. In some embodiments, the internal volume of the perfusion bioreactor is about 100 L to about 500 L. In some embodiments, the internal volume of the perfusion bioreactor is about 100 L to about 400 L. In some embodiments, the internal volume of the perfusion bioreactor is about 100 L to about 300 L. In some embodiments, the internal volume of the perfusion bioreactor is about 100 L to about 200 L.

In some embodiments, the internal volume of the perfusion bioreactor is about 500 L to about 1000 L. In some embodiments, the internal volume of the perfusion bioreactor is about 500 L to about 900 L. In some embodiments, the internal volume of the perfusion bioreactor is about 500 L to about 800 L. In some embodiments, the internal volume of the perfusion bioreactor is about 500 L to about 700 L. In some embodiments, the internal volume of the perfusion bioreactor is about 500 L to about 600 L. Any other suitable internal volume can also be used in the method descried herein.

In some embodiments, the volume of the liquid culture medium is about 1 L to about 1000 L. In some embodiments, the volume of the liquid culture medium is about 1 L to about 900 L. In some embodiments, the volume of the liquid culture medium is about 1 L to about 800 L. In some embodiments, the volume of the liquid culture medium is about 1 L to about 700 L. In some embodiments, the volume of the liquid culture medium is about 1 L to about 600 L. In some embodiments, the volume of the liquid culture medium is about 1 L to about 500 L. In some embodiments, the volume of the liquid culture medium is about 1 L to about 400 L. In some embodiments, the volume of the liquid culture medium is about 1 L to about 300 L. In some embodiments, the volume of the liquid culture medium is about 1 L to about 200 L. In some embodiments, the volume of the liquid culture medium is about 1 L to about 100 L. In some embodiments, the volume of the liquid culture medium is about 1 L to about 90 L. In some embodiments, the volume of the liquid culture medium is about 1 L to about 80 L. In some embodiments, the volume of the liquid culture medium is about 1 L to about 70 L. In some embodiments, the volume of the liquid culture medium is about 1 L to about 60 L. In some embodiments, the volume of the liquid culture medium is about 1 L to about 50 L. In some embodiments, the volume of the liquid culture medium is about 1 L to about 40 L. In some embodiments, the volume of the liquid culture medium is about 1 L to about 30 L. In some embodiments, the volume of the liquid culture medium is about 1 L to about 20 L. In some embodiments, the volume of the liquid culture medium is about 1 L to about 10 L. In some embodiments, the volume of the liquid culture medium is about 1 L to about 9 L. In some embodiments, the volume of the liquid culture medium is about 1 L to about 8 L. In some embodiments, the volume of the liquid culture medium is about 1 L to about 7 L. In some embodiments, the volume of the liquid culture medium is about 1 L to about 6 L. In some embodiments, the volume of the liquid culture medium is about 1 L to about 5 L.

In some embodiments, the volume of the liquid culture medium is about 5 L to about 1000 L. In some embodiments, the volume of the liquid culture medium is about 5 L to about 900 L. In some embodiments, the volume of the liquid culture medium is about 5 L to about 800 L. In some embodiments, the volume of the liquid culture medium is about 5 L to about 700 L. In some embodiments, the volume of the liquid culture medium is about 5 L to about 600 L. In some embodiments, the volume of the liquid culture medium is about 5 L to about 500 L. In some embodiments, the volume of the liquid culture medium is about 5 L to about 400 L. In some embodiments, the volume of the liquid culture medium is about 5 L to about 300 L. In some embodiments, the volume of the liquid culture medium is about 5 L to about 200 L. In some embodiments, the volume of the liquid culture medium is about 5 L to about 100 L. In some embodiments, the volume of the liquid culture medium is about 5 L to about 90 L. In some embodiments, the volume of the liquid culture medium is about 5 L to about 80 L. In some embodiments, the volume of the liquid culture medium is about 5 L to about 70 L. In some embodiments, the volume of the liquid culture medium is about 5 L to about 60 L. In some embodiments, the volume of the liquid culture medium is about 5 L to about 50 L. In some embodiments, the volume of the liquid culture medium is about 5 L to about 40 L. In some embodiments, the volume of the liquid culture medium is about 5 L to about 30 L. In some embodiments, the volume of the liquid culture medium is about 5 L to about 20 L. In some embodiments, the volume of the liquid culture medium is about 5 L to about 10 L.

In some embodiments, the volume of the liquid culture medium is about 10 L to about 1000 L. In some embodiments, the volume of the liquid culture medium is about 10 L to about 900 L. In some embodiments, the volume of the liquid culture medium is about 10 L to about 800 L. In some embodiments, the volume of the liquid culture medium is about 10 L to about 700 L. In some embodiments, the volume of the liquid culture medium is about 10 L to about 600 L. In some embodiments, the volume of the liquid culture medium is about 10 L to about 500 L. In some embodiments, the volume of the liquid culture medium is about 10 L to about 400 L. In some embodiments, the 10 volume of the liquid culture medium is about 10 L to about 300 L. In some embodiments, the volume of the liquid culture medium is about 10 L to about 200 L. In some embodiments, the volume of the liquid culture medium is about 10 L to about 100 L. In some embodiments, the volume of the liquid culture medium is about 10 L to about 90 L. In some embodiments, the volume of the liquid culture medium is about 10 L to about 80 L. In some embodiments, the volume of the liquid culture medium is about 10 L to about 70 L. In some embodiments, the volume of the liquid culture medium is about 10 L to about 60 L. In some embodiments, the volume of the liquid culture medium is about 10 L to about 50 L. In some embodiments, the volume of the liquid culture medium is about 10 L to about 40 L. In some embodiments, the volume of the liquid culture medium is about 10 L to about 30 L. In some embodiments, the volume of the liquid culture medium is about 10 L to about 20 L.

In some embodiments, the volume of the liquid culture medium is about 50 L to about 1000 L. In some embodiments, the volume of the liquid culture medium is about 50 L to about 900 L. In some embodiments, the volume of the liquid culture medium is about 50 L to about 800 L. In some embodiments, the volume of the liquid culture medium is about 50 L to about 700 L. In some embodiments, the volume of the liquid culture medium is about 50 L to about 600 L. In some embodiments, the volume of the liquid culture medium is about 50 L to about 500 L. In some embodiments, the volume of the liquid culture medium is about 50 L to about 400 L. In some embodiments, the volume of the liquid culture medium is about 50 L to about 300 L. In some embodiments, the volume of the liquid culture medium is about 50 L to about 200 L. In some embodiments, the volume of the liquid culture medium is about 50 L to about 100 L.

In some embodiments, the volume of the liquid culture medium is about 100 L to about 1000 L. In some embodiments, the volume of the liquid culture medium is about 100 L to about 900 L. In some embodiments, the volume of the liquid culture medium is about 100 L to about 800 L. In some embodiments, the volume of the liquid culture medium is about 100 L to about 700 L. In some embodiments, the volume of the liquid culture medium is about 100 L to about 600 L. In some embodiments, the volume of the liquid culture medium is about 100 L to about 500 L. In some embodiments, the volume of the liquid culture medium is about 100 L to about 400 L. In some embodiments, the volume of the liquid culture medium is about 100 L to about 300 L. In some embodiments, the volume of the liquid culture medium is about 100 L to about 200 L.

In some embodiments, the volume of the liquid culture medium is about 500 L to about 1000 L. In some embodiments, the volume of the liquid culture medium is about 500 L to about 900 L. In some embodiments, the volume of the liquid culture medium is about 500 L to about 800 L. In some embodiments, the volume of the liquid culture medium is about 500 L to about 700 L. In some embodiments, the volume of the liquid culture medium is about 500 L to about 600 L.

Any other suitable volume of the liquid culture medium can also be used in the method descried herein.

The mammalian cell used in the method described herein can be any suitable mammalian cell known in the art. In some embodiments, the mammalian cell is a mammalian cell line. Cell lines that can be used for the production of various products such as recombinant proteins and antibodies are known in the art. In some embodiments, the mammalian cell line is a Chinese Hamster Ovarian (CHO) cell line.

The culturing of the mammalian cells can be performed in any suitable cell culture medium. Suitable culture medium for bioprocessing are known in the art.

In one aspect, provided herein is a method of culturing a mammalian cell in a perfusion bioreactor comprising: (a) providing a perfusion bioreactor having a DO probe at a fixed orientation and containing a mammalian cell in a liquid culture medium; and (b) culturing the mammalian cell using the perfusion bioreactor under conditions sufficient to achieve an optimal DO level, wherein the perfusion bioreactor has an internal volume for a certain bioprocess.

Also provided herein is a method of culturing a cell in a perfusion bioreactor comprising: (a) providing a perfusion bioreactor having a DO probe at a fixed orientation and containing a cell in a liquid culture medium; and (b) culturing the cell using the perfusion bioreactor under conditions sufficient to achieve an optimal DO level, wherein the perfusion bioreactor has an internal volume for a certain bioprocess.

Any fixed orientation of the DO probe described herein can be used in any of the methods described herein.

In some embodiments, the optimal DO level is about 20% to about 90%, about 20% to about 80%, about 20% to about 70%, about 20% to about 60%, about 20% to about 50%, about 20% to about 40%, about 20% to about 30%, about 30% to about 90%, about 30% to about 80%, about 30% to about 70%, about 30% to about 60%, about 30% to about 50%, about 30% to about 40%, about 40% to about 90%, about 40% to about 80%, about 40% to about 70%, about 40% to about 60%, about 40% to about 50%, about 50% to about 90%, about 50% to about 80%, about 50% to about 70%, about 50% to about 60%, 60% to about 90%, about 60% to about 80%, about 60% to about 70%, 70% to about 90%, about 70% to about 80%, or about 80% to about 90%. In some embodiments, the optimal DO level is about 20% to about 40%.

In one aspect, provided herein is a method of culturing a mammalian cell in a perfusion bioreactor comprising: (a) providing a perfusion bioreactor having a DO probe at a fixed angle of about 60-70 degrees with a 0-degree to about a 10-degree rotation and containing a mammalian cell in a liquid culture medium; and (b) culturing the mammalian cell using the perfusion bioreactor under conditions sufficient to achieve a DO level of about 20% to about 40%, wherein the perfusion bioreactor has an internal volume of about 5 L to about 1000 L.

Also provided herein is a method of culturing a cell in a perfusion bioreactor comprising: (a) providing a perfusion bioreactor having a DO probe at a fixed angle of about 60-70 degrees with a 0-degree rotation and containing a cell in a liquid culture medium; and (b) culturing the cell using the perfusion bioreactor under conditions sufficient to achieve a DO level of about 20% to about 40%, wherein the perfusion bioreactor has an internal volume of about 5 L to about 1000 L.

In some embodiments, the internal volume of the perfusion bioreactor is about 5 L to about 100 L. In some embodiments, the internal volume of the perfusion bioreactor is about 5 L to about 15 L. In some embodiments, the internal volume of the perfusion bioreactor is about 100 L to about 500 L. Any other suitable internal volume can also be used in the method descried herein.

In some embodiments, the conditions sufficient to achieve the optimal DO level (e.g., about 20% to about 40%) includes a volumetric input power of about 20 W/m³ to about 90 W/m³, about 20 W/m³ to about 80 W/m³, about 20 W/m³ to about 70 W/m³, about 20 W/m³ to about 60 W/m³, about 20 W/m³ to about 50 W/m³, about 20 W/m³ to about 40 W/m³, about 20 W/m³ to about 30 W/m³, about 30 W/m³ to about 90 W/m³, about 30 W/m³ to about 80 W/m³, about 30 W/m³ to about 70 W/m³, about 30 W/m³ to about 60 W/m³, about 30 W/m³ to about 50 W/m³, about 30 W/m³ to about 40 W/m³, about 40 W/m³ to about 90 W/m³, about 40 W/m³ to about 80 W/m³, about 40 W/m³ to about 70 W/m³, about 40 W/m³ to about 60 W/m³, about 40 W/m³ to about 50 W/m³, about 50 W/m³ to about 90 W/m³, about 50 W/m³ to about 80 W/m³, about 50 W/m³ to about 70 W/m³, about 50 W/m³ to about 60 W/m³, about 60 W/m³ to about 90 W/m³, about 60 W/m³ to about 80 W/m³, about 60 W/m³ to about 70 W/m³, about 70 W/m³ to about 90 W/m³, about 70 W/m³ to about 80 W/m³, or about 80 W/m³ to about 90 W/m³. In some embodiments, the conditions sufficient to achieve the optimal DO level (e.g., about 20% to about 40%) includes a volumetric input power of about 36 W/m³ to about 65 W/m³.

In some embodiments, the cell is a mammalian cell. In some embodiments, the cell is a non-mammalian cell. The mammalian cell used in the method described herein can be any suitable mammalian cell known in the art. In some embodiments, the mammalian cell is a mammalian cell line. Cell lines that can be used for the production of various products such as recombinant proteins and antibodies are known in the art. In some embodiments, the mammalian cell line is a Chinese Hamster Ovarian (CHO) cell line.

Any suitable perfusion bioreactor can be used in the methods described herein. In some embodiments, the perfusion bioreactor is a flexible bag bioreactor. In some embodiments, the perfusion bioreactor is a benchtop bioreactor. In some embodiments, the perfusion bioreactor is a stainless steel bioreactor. In some embodiments, the perfusion bioreactor is a single-use bioreactor. In some embodiments, the perfusion bioreactor allows the adjustment of the fixed angle and/or degree of rotation of the DO probe. In some embodiments, the perfusion bioreactor allows the rotation of the DO probe positioned at a fixed angle. In some embodiments, the perfusion bioreactor allows the rotation of the DO probe and the adjustment of the fixed angle of the DO probe.

### EXAMPLES

The invention is further described in the following examples, which do not limit the scope of the invention described in the claims.

### Materials and Methods

### Bioreactor and Cell-Retention Device

The pilot scale was conducted with Thermo Fisher's single-use bioreactor connected to Repligen's ATF6 (Alternating Tangential Filtration) as a cell-retention device. The benchtop scale was conducted with Broadley James's 5 L bioreactor connected to Repligen's ATF2.

### Dissolved Oxygen Probe

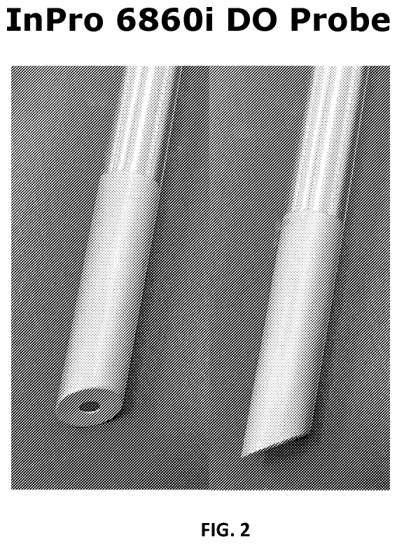

FIG. 2 shows an example of a commercially available DO probe InPro6860i (Mettler Toledo). The DO of all cultures was measured with Mettler Toledo's InPro6860i. It is an optical DO probe. The probe is capped with an OptoCap BT02T, which has a PTFE coating to resist bubble coagulation. Additionally, the tip of the OptoCap is angled at 30° leading to a potential for anisotropy when it comes to the rotation of the probe within the bioreactor. This probe was selected for use in this process due to difficulties accurately measuring the DO at high cell densities with our historical polarographic probe.

### Operating Parameters

All cultures for these data were inoculated at 0.5-1.0×10⁶ cells/mL. Perfusion was started at 1 RV/day on day 1 and increased to 2 RV/day on day 5. Biomass concentration was controlled by capacitance measurement and a cell bleed. Other parameters were controlled at standard cell culture settings. Proprietary chemically defined medium was used for all runs.

### Example 1: High DO Impact on Culture Performance at Pilot Scale

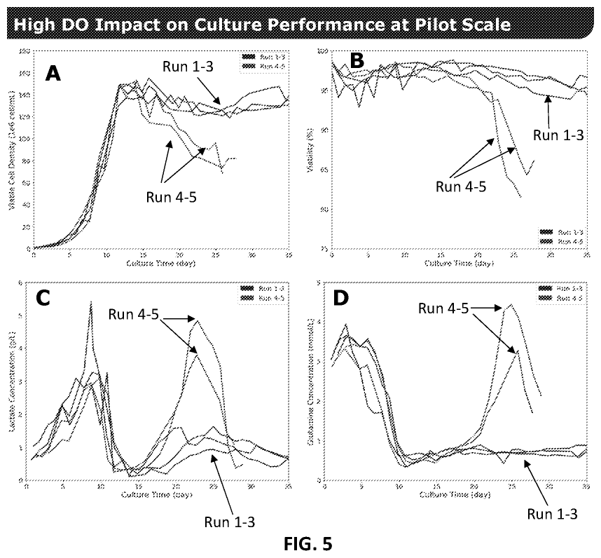

The first three pilot scale batches that were run for this project had the DO probes oriented with the probe window facing rightwards (with an angle of about 60-70 degrees as illustrated in FIGS. 3A-3E and a rotation of about 90 degrees in the direction as illustrated in FIGS. 4A-4B). To harmonize with other perfusion projects, the probes were reoriented to face upwards (with an angle of about 60-70 degrees as illustrated in FIGS. 3A-3E and a rotation of about 0 degree as illustrated in FIGS. 4A-4B). This was predicted to have a negligible impact on the DO measurement based on data collected at the time. However, two batches run in parallel after this change exhibited unusual behavior. FIGS. 5A-5D compare the performance of Runs 4 and 5 to the original three batches. Soon after reaching peak VCD, the cultures experienced a dramatic decline in growth and metabolic efficiency. The cells shifted back towards lactate production, peaking at 4-5 g/L with approximately half of the glucose being consumed getting diverted to lactate accumulation. The bleed rate ultimately stopped as the culture couldn't maintain the capacitance setpoint and both runs were terminated early due to low viability.

### Example 2: Effect of Probe Orientation on DO Signal and DO Setpoint of Pilot Scale Batches Depending on Orientation

While investigating the root cause of the discrepancies in Runs 4 and 5, it was noted that the DO probe actually measured approximately 30 percentage points lower when oriented upwards (with an angle of about 60-70 degrees as illustrated in FIGS. 3A-3E and a rotation of about 0 degree as illustrated in FIGS. 4A-4B). After further studies, the data in Table 1 was generated. This summarizes the relationship between volumetric power input from the impeller and the differential measurement between when the probe is oriented up (with an angle of about 70 degrees as illustrated in FIGS. 3A-3E and a rotation of about 0 degree as illustrated in FIGS. 4A-4B) or right (with an angle of about 60-70 degrees as illustrated in FIGS. 3A-3E and a rotation of about 90 degrees in the direction as illustrated in FIGS. 4A-4B). Table 2 then compares the DO setpoints of several pilot scale batches at the two probe orientations. Based on relatively lower noise in the signal and historical precedent, it was decided to continue forward with the upwards probe orientation. As can be seen in Table 2, Run 3 was shifted to a comparable DO setpoint to Runs 4 and 5 and did not exhibit similar behavior. Nonetheless, the dysfunctional performance of Runs 4 and 5 and the following investigations led to the hypothesis that the combination of high DO with an insufficient mixing environment induced the metabolic shifts observed.

TABLE 1
Comparison of the difference in DO measurement
between when the probe is facing right or up.

| Volumetric Power Input | DO Measurement |
|---|---|
| (W/m³) | Differential (%)* |
| 36 | 30 |
| 55 | 15 |
| 65 | 10 |

*Differential is the right value minus the up value

TABLE 2
Comparison of pilot scale batches by their volumetric power
input and DO setpoint when facing either right or up.

| | Volumetric | Rightward | Upward |
|---|---|---|---|
| Run # | Power Input (W/m³) | DO (%)** | DO (%)** |
| 1 | 25 | 50 | 20 |
| 2 | 36 | 50 | 20 |
| 3 | 36 | 50-85 | 20-55 |
| 4 + 5 | 36 | 90 | 60 |
| 6 | 55-65 | 50-60 | 40-50 |
| 7 | 65 | 50 | 40 |

**Bolded values mean the bioreactor was controlled off a probe in that orientation

### Example 3: Benchtop DO Range Study

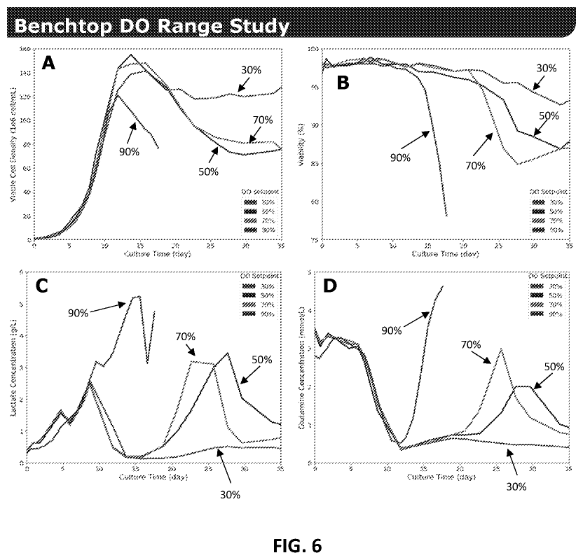

Benchtops were planned to test DO setpoints ranging from 30 to 90% to better characterize the culture's response. This benchtop model was operated a volumetric power input of 90 W/m³, which is far higher than any of the pilot scale runs. FIGS. 6A-6D compares a few parameters between the conditions. The cultures performed comparably through the growth phase, but as the conditions at 30 to 70% DO shifted towards lactate re-consumption the 90% DO culture continued to accumulate lactate and growth rapidly declined. The 90% DO culture was terminated early after viability had declined under 80%. Soon after reaching peak VCD, the 50 and 70% conditions then entered a similar state of low growth and metabolic dysfunction as the pilot scale. Unlike the 90% DO condition both cultures were able to recover without as much of a drop in viability.

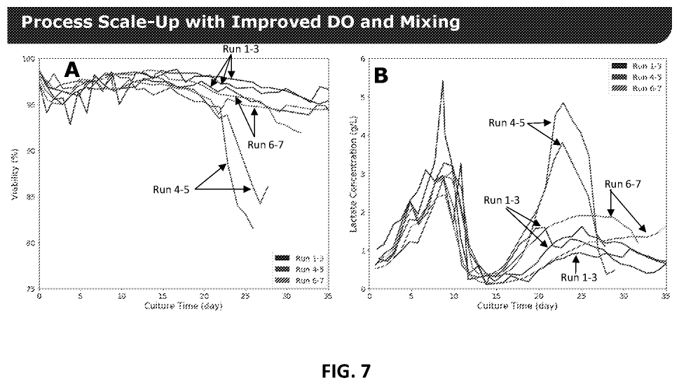

Based on the observations from previous batches, the pilot scale bioreactor was re-designed for improved mixing, increasing the volumetric power input from 36 to 65 W/m³. The DO setpoint was lowered to 40% with an upwards DO probe. FIGS. 7A-7B compare the performance of Runs 6 and 7, which were conducted with these changes in place, to the previous pilot scale runs. These runs were able to maintain culture health and metabolism more in line with the original three batches. Table 3 characterizes the degree of risk to the process at various DO setpoints as we have observed from these data.

TABLE 3
Process risk guide for ranges of DO setpoints

| DO Setpoint (%) | Process Risk |
|---|---|
| 20-40 | Safe |
| 50-70 | Caution |
| >90 | High Risk |

These results show that lowering the DO setpoint and re-designing the pilot scale system to accommodate more effective mixing enabled a more robust scale-up.

In summary, a change in the procedure for probe operation in the pilot scale bioreactor (i.e., the orientation of probe orientation) caused a significant shift in the DO measurement and resulted in the early termination of two batches if the DO setpoints were not adjusted accordingly. Scale-down experiments confirmed that higher DO increases the risk of inducing an unfavorable metabolic state. Therefore, DO measurement and control at different bioreactor scales is critical in eliminating such undesired results and carefully selecting an orientation of DO probe to eliminate the falsely increased DO measurement is needed for optimal outcomes of production.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of culturing a mammalian cell in a perfusion bioreactor, wherein the method comprises:
(a) placing a dissolved oxygen (DO) probe in the perfusion bioreactor comprising a liquid at a plurality of different fixed orientations;
(b) measuring the DO level using the DO probe at each of the plurality of different fixed orientations;
(c) selecting a fixed orientation for the DO probe in the bioreactor based on the measured DO levels in step (b), wherein the selected fixed orientation does not demonstrate measurement of falsely increased DO levels as determined by analysis of the measured DO levels in step (b); and
(d) culturing a mammalian cell in a liquid culture medium in the perfusion bioreactor having the DO probe positioned at the selected fixed orientation.

2. A method of selecting a fixed orientation for a dissolved oxygen (DO) probe in a perfusion bioreactor, the method comprising:
(a) placing the DO probe in the perfusion bioreactor comprising a liquid at a plurality of different fixed orientations;
(b) measuring the DO level using the DO probe at each of the plurality of different fixed orientations; and
(c) selecting a fixed orientation for the DO probe in the bioreactor based on the measured DO levels in step (b), wherein the selected fixed orientation does not demonstrate measurement of falsely increased DO levels as determined by analysis of the measured DO levels in step (b).

3. The method of claim 2, wherein the method further comprises:
(d) performing a plurality of mammalian cell culture runs in the perfusion bioreactor having the DO probe positioned at the selected fixed orientation, where each of the plurality of mammalian cell culture runs has a different measured DO level as measured by the DO probe;
(e) detecting one or more of viable cell density, lactate production, and recombinant protein production by cells in each mammalian cell culture run; and
(f) selecting a DO level for additional mammalian cell culture runs in the perfusion bioreactor based on one or more of the detected viable cell density, lactate production, and recombinant protein production in step (e) for each mammalian cell culture run.

4. The method of claim 3, wherein the method further comprises culturing a mammalian cell in a perfusion bioreactor having a DO probe at the selected fixed orientation in a liquid culture medium under conditions that result in the selected DO level.

5. The method of claim 1, wherein the DO probe is a rod-shaped DO probe.

6. The method of claim 1, wherein the DO probe comprises an optical sensor.

7. The method of claim 6, wherein the DO probe has a base and a tip, wherein the optical sensor is located at the tip-end of the DO probe.

8. The method of claim 1, wherein the fixed orientation is identified by a fixed angle of the DO probe.

9. The method of claim 1, wherein the plurality of different fixed orientations is a plurality of different fixed angles between about 0 degrees and about 180 degrees.

10.-11. (canceled)

12. The method of claim 7, wherein the tip of the DO probe is tilted or slanted.

13. The method of claim 12, wherein each of the plurality of different fixed orientations is identified by a fixed angle of the DO probe and degree of rotation of the DO probe along the longitudinal axis of the DO probe.

14. The method of claim 13, wherein each of the plurality of different fixed orientations have a shared fixed angle of the DO probe and different degrees of rotation of the DO probe along the longitudinal axis of the DO probe.

15. The method of claim 13, wherein the plurality of different fixed orientations are a plurality of different degrees of rotation of the DO probe along the longitudinal axis of the DO probe in a plurality of different fixed angles.

16.-18. (canceled)

19. The method of claim 6, wherein the selected fixed orientation of the DO probe reduces accumulation of air bubbles on or near the sensor of the DO probe.

20. The method of claim 4, wherein one or more of volumetric input power, culturing temperature, and culture volume are adjusted to achieve the selected DO level.

21.-22. (canceled)

23. The method of claim 1, wherein the mammalian cell is a mammalian cell line.

24. (canceled)

25. A method of culturing a mammalian cell in a perfusion bioreactor comprising:
(a) providing a perfusion bioreactor having a DO probe at a fixed orientation with a fixed angle of about 60-70 degrees and about a 0-degree to 10-degree rotation and containing a mammalian cell in a liquid culture medium; and
(b) culturing the mammalian cell using the perfusion bioreactor under conditions sufficient to achieve a DO level of about 20% to about 40%,
wherein the perfusion bioreactor has an internal volume of about 5 L to about 1000 L.

26. The method of claim 25, wherein the conditions sufficient to achieve a DO level of about 20% to about 40% comprises a volumetric input power of about 30 W/m³ to about 90 W/m³.

27. The method of claim 26, wherein the conditions sufficient to achieve a DO level of about 20% to about 40% comprises a volumetric input power of about 36 W/m³ to about 65 W/m³.

28.-33. (canceled)

34. The method of claim 25, wherein the mammalian cell is a mammalian cell line.

35. (canceled)
